(19) United States
(12) Patent Application Publication
(10) Pub. No.: US 2018/0209911 A1
(43) Pub. Date: Jul. 26, 2018

(54) PH DISTRIBUTION MEASUREMENT IN A POROUS MATERIAL MICRO-STRUCTURE METHOD AND APPARATUS

(71) Applicants: Svetozar Katuscak, Port St. Lucie, FL (US); {hacek over (S)}tefan Vodný, Bratislava (SK); Katarína Vizárová, Bratislava (SK)

(72) Inventors: Svetozar Katuscak, Port St. Lucie, FL (US); {hacek over (S)}tefan Vodný, Bratislava (SK); Katarína Vizárová, Bratislava (SK)

(21) Appl. No.: 15/613,115

(22) Filed: Jun. 2, 2017

Related U.S. Application Data
(60) Provisional application No. 62/450,303, filed on Jan. 25, 2017.

(51) Int. Cl. G01N 21/80; G01N 33/34

(52) U.S. Cl. CPC G01N 21/80; G01N 33/34; G01N 33/346

(57) ABSTRACT

A method and apparatus for pH distribution measurement in a porous material micro-structure is disclosed. In one embodiment, a method of measurement of pH distribution within the micro-structure of a porous material, such as cell, plant, fiber or cellulose material, paper or cultural object of porous materials (next material) is disclosed. The method comprises the steps of (in any order) preparing a microscopic sample of the porous material at a selected magnification; selecting an elementary measured area of the microscopic sample or microimage; microimaging the elementary measured area; measuring one or more pH characteristic parameters and pH from the elementary measured area, and measuring a correlation between the one or more characteristic optical parameters from the elementary measured area and the pH of from the elementary measured area to obtain a correlated microscopic pH value and a microscopic characteristic optical parameter value and their distribution within the porous material micro-structure.

### PRIORITY

This application is an U.S. Utility patent application filed under 111(c) claiming priority to U.S. Provisional Patent application No. 62/450,303, filed on Jun. 2, 2016, the entire disclosure of which is hereby relied on for all purposes and is incorporated by reference into this application its entirety, including the drawings.

### FIELD OF TECHNOLOGY

The present disclosure relates to a measuring apparatus and method. More particularly, the present invention relates to an apparatus and method for measuring pH, μpH and μpH distribution, as well as related distribution of alkali compounds, and alkaline reserves in porous materials, cellular materials, or cellulose materials and objects, such as books or paper documents intended to be saved for future generations, plant and food materials, biomaterials, or biological materials or preparations. This invention is also aimed at identification of pockets of acidification in deacidified materials, identifying acidification in the micro-structure of materials, and identification of incomplete deacidification of books and paper.

A method and apparatus to conservation of a multi-functional conservation apparatus suitable for books or paper documents intended to be saved for future generations.

### BACKGROUND

National libraries, other preserving libraries, archives, museums, and private companies around the world stabilize documents, books, and archival documents against rapid statistical cleavage of cellulose which takes place in an acidic environment. These institutions implement neutralization of acids in these materials, and create alkaline reserves, using mass deacidification technology.

When treating acids in these materials, consider the type and nature of the acids that are found in the materials. Acids occur at a macromolecular level, and in the micro-structure of the paper.

There are big differences in quality and type of deacidification processes. Some processes are effective against macro-distribution of pH in the material, book, or other objects, while others are effective against micro-distribution. The differences between the processes can be caused by occurrence of uneven or incomplete acidification. The differences between the processes are in extending the lifetime and efficiency in terms of conservation of mechanical, physical, and chemical properties, information, and functions of the carrier of information and documents.

To elaborate, cellulose degradation takes place at the level of macromolecules, meaning a range of 1 to 100 Å, mainly by oxidation, such as by oxygen and acids, because acidification occurs within supramolecular structures. If the stabilizing compounds are alkalies used for deacidification, they must penetrate through all supramolecular structures. If, for example, the alkali stays on the surface of the supramolecular structure, then the cellulose macromolecules can remain acidic, the cells remain acidic, and the cell walls, fibrils, and microfibrils can stay acidic. In the acidic part of material continues degradation by oxidation, acid hydrolysis, and/or continually forming new acids and acid environments, this further decreases the pH of macromolecules.

The neutralization, alkalization in the industrial alkaline paper production, or post-production deacidification of the acid paper cure the symptoms, not the cause of acidification or acid formation. From this point of view, the neutralization and deacidification used in both the alkaline paper production, or in the post-production stabilization and conservation, is still an incomplete solution of stabilization of cell materials against natural, lawful, and continuing oxidation and acids formation.

### SUMMARY

A method and apparatus for pH distribution measurement in a porous material micro-structure is disclosed.

In one aspect, a method of measurement of pH distribution within the micro-structure of a porous material, such as cell, plant, fiber or cellulose material, paper or cultural object of porous materials (next material) is disclosed. The method comprises the steps of (in any order):
a. preparing a microscopic sample of the porous material at a selected magnification;
b. selecting an elementary measured area of the microscopic sample or microimage;
c. microimaging the elementary measured area;
d. measuring one or more pH characteristic parameters and pH from the elementary measured area, and measuring a correlation between the one or more characteristic optical parameters from the elementary measured area and the pH of from the elementary measured area to obtain a correlated microscopic pH value and a microscopic characteristic optical parameter value and their distribution within the porous material micro-structure; whereas the pH characteristic parameter (CP) is a parameter of porous material microsample or macrosample correlating with the measured pH, pH distribution (pHd), micro-pH (μpH) or micro-pH distribution (μpHd) in the micro-structure of the sample measured, and invariant, or possibly minimally depending on the other factors of variability of pH, and pHd measurement which are to be eliminated or minimize; such factors of variability are porous morphological structure, defects, inter fibre or intra fibre pores, presence of lumens, sort of fibres, tissues inside, sort of material or raw materials used, such as sort of wood used for pulping, conservation process, whether the material was deacidified or not, modified, or otherwise treated, whether its average pH is alkaline or acid, and other chemical or physical properties not correlating with pH;
e. preparing a macroscopic sample of the porous material;
f. measuring and correlating one or more pH-characteristic parameters and pH of a macroscopic sample of the porous material to select the best correlating CP, and to obtain a correlated macroscopic pH value from the macroscopic pH characteristic optical parameter value; and
g. measuring and correlating one or more pH characteristic parameters and pH of the porous material microscope sample at various magnifications of interest to select the best correlating CP, and to obtain a correlated macroscopic pH value from the macroscopic pH characteristic optical parameter value.

An elementary measured area may be approximately 0.1 to 5 microns. Characteristic optical parameters may be selected from a group Magnesium-, Aluminum-, Zinc-, Calcium EDS signals, color parameters, CIE total color difference, reflectance, and/or a combination thereof.

The method may also include additional steps including, but not limited to:
h. applying a subcritical no migration or sub-migration cyclic impregnation of the porous material using the aqueous solution of pH indicator, wherein the subcritical no migration or sub-migration cyclic impregnation further comprises depositing the pH indicator solution aerosol to the surface of the elementary measured area and macroscopic sample; and
i. applying colorimetric control of the subcritical, no migration or sub-migration cyclic impregnation by measuring one or more pH characteristic optical parameters at two different positions of sample to measure, control and eliminate the migration of alkali, acids of pH distribution, with advantage using the apparatus according to claims 5-9 during, the subcritical time is and using subcritical amount of deposited water or aqueous solution at one cycle ms, followed by drying the material.

One or more characteristic pH characteristic optical parameters may be characterized and/or measured by Scanning Electron Microscopy/Energy Dispersive X-Ray Spectroscopy (SEM/EDS) and/or Scanning Electron Microscopy/Wavelength Dispersive Spectroscopy (SEM/WDS). One or more pH characteristic parameter may be characterized in the CIE tristimulárne or spectral characteristics of the optical properties in the visible spectrum of 400-700 nm of paper and/or pH indicator, indicating substance. The method may be characterized in that the sample impregnation by pH indicator color by SAT performed after the neutralization, deacidification, and conservation or the cell material.

An apparatus for measuring pH in micro-structure of a material may also be contemplated according to this aspect. The apparatus may include (1) atomizer or nebulizer, or aerosol generators; (2) a microscope, mobile or smartphone microscope; and (3) a micromanipulator with tools for preparation and modification of the microscopic preparation from porous materials, such as cultural material or object. The tools can be a cylindrical or rectangular blade for non-destructive or quasi non-destructive sampling, micro abrasion tool, and sample modification such as splitting, scanning and automated image analyses apparatus, or sheet splitting with a heat seal lamination technique apparatus. The apparatus for the pH measurement in material micro-structure in this aspect and others may comprise parts according to the Example 7.

In another aspect, a method of measurement of pH distribution within a micro-structure of a porous material includes: preparing a microscopic sample of the porous material at a selected magnification; selecting an elementary measured area of the microscopic sample, microimaging the elementary measured area, measuring a pH characteristic parameters (CP) and pH from the elementary measured area, measuring a correlation between the pH characteristic parameters from the elementary measured area and a pH of from the elementary measured area to obtain a correlated microscopic pH value and a microscopic characteristic optical parameter value and a distribution within a micro-structure of the porous material, whereas each of the pH characteristic parameters is a parameter of at least one of a porous material microsample and a porous material macrosample correlating with at least one of a measured pH, a pH distribution (pHd), a micro-pH (μpH), a micro-pH distribution (μpHd) in the micro-structure of the sample measured, an invariant, and possibly minimally depending on other factors of variability of pH, and pHd measurement which are an attempt to eliminate such factors of variability are porous morphological structure, defects, at least one of inter fibre and intra fibre pores, presence of lumens, sort of fibres, tissues inside, sort of material used, such as sort of wood used for pulping, conservation process, whether material was deacidified, modified, and otherwise treated, whether its average pH is alkaline, whether its average pH is acid, and other chemical and physical properties not correlating with pH.

The method in this other aspect also includes preparing a macroscopic sample of the porous material, measuring and correlating pH-characteristic parameters and pH of a macroscopic sample of the porous material to select the best correlating CP, and to obtain a correlated macroscopic pH value from a macroscopic pH characteristic optical parameter value, and measuring and correlating pH characteristic parameters and pH of the porous material microscope sample at various magnifications of interest to select the best correlating CP, and to obtain a correlated macroscopic pH value from the macroscopic pH characteristic optical parameter value. An elementary measured area may be approximately 0.1 to 5 microns.

In yet another aspect, an apparatus to measure pH distribution within a micro-structure of a porous material includes a nebulizer to producing a fine spray of liquid, a microscope to magnify the porous material at least several hundred times, and a micromanipulator with a set of tools to prepare and modify a microscopic preparation from the porous material. The set of tools perform a set of functions including preparing a microscopic sample of the porous material at a selected magnification, selecting an elementary measured area of the microscopic sample, microimaging the elementary measured area, measuring a pH characteristic parameters (CP) and pH from the elementary measured area, measuring a correlation between the pH characteristic parameters from the elementary measured area and a pH of from the elementary measured area to obtain a correlated microscopic pH value and a microscopic characteristic optical parameter value and a distribution within a micro-structure of the porous material.

Each of the pH characteristic parameters is a parameter of at least one of a porous material microsample and a porous material macrosample correlating with at least one of a measured pH, a pH distribution (pHd), a micro-pH (μpH), a micro-pH distribution (μpHd) in the micro-structure of the sample measured, an invariant, and possibly minimally depending on other factors of variability of pH, and pHd measurement which are attempted to be eliminated such factors of variability are porous morphological structure, defects, at least one of inter fibre and intra fibre pores, presence of lumens, sort of fibres, tissues inside, sort of material used, such as sort of wood used for pulping, conservation process, whether material was deacidified, modified, and otherwise treated, whether its average pH is alkaline, whether its average pH is acid, and other chemical and physical properties not correlating with pH,

The set of tools may perform additional functions including preparing a macroscopic sample of the porous material, measuring pH-characteristic parameters and pH of a macroscopic sample of the porous material to select the best correlating CP, and to obtain a correlated macroscopic pH value from a macroscopic pH characteristic optical parameter value, measuring pH characteristic parameters and pH of the porous material microscope sample at various magnifications of interest to select the best correlating CP, and to obtain a correlated macroscopic pH value from the macroscopic pH characteristic optical parameter value, correlating pH-characteristic parameters and pH of the macroscopic sample of the porous material to select the best correlating CP, and to obtain the correlated macroscopic pH value from the macroscopic pH characteristic optical parameter value, and correlating pH characteristic parameters and pH of the porous material microscope sample at various magnifications of interest to select the best correlating CP, and to obtain a correlated macroscopic pH value from the macroscopic pH characteristic optical parameter value.

The nebulizer may be an aerosol generator and an atomizer to reduce the liquid into the fine spray. The set of tools may include a cylindrical and/or a rectangle blade for at least one of a non-destructive and a quasi non-destructive sampling, micro abrasion tool, and sample modification such as a splitting, a scanning and a automated image analysis apparatus, and sheet splitting with a heat seal lamination technique apparatus. An elementary measured area may be approximately 0.1 to 5 microns. The characteristic optical parameters may be selected from a group comprising at least one of a Magnesium-, Aluminum-, Zinc-, Calcium EDS signals, color parameters, CIE total color difference, reflectance, and a combination thereof.

The set of tools may perform a set of functions including applying at least one of a subcritical no migration and a sub-migration cyclic impregnation of the porous material using the aqueous solution of pH indicator, wherein the at least one of the subcritical no migration and the sub-migration cyclic impregnation further comprises depositing the pH indicator solution aerosol to the surface of the elementary measured area and macroscopic sample, and applying a colorimetric control of the subcritical, at least one of the no migration and the sub-migration cyclic impregnation by measuring pH characteristic optical parameters at two different positions of sample to measure, control and eliminate the migration of alkali, acids of pH distribution, with advantage using the apparatus according to claims 5-9 during, the subcritical time is and using subcritical amount of deposited aqueous solution at one cycle ms, followed by drying the material.

The characteristic pH characteristic optical parameters may be characterized and/or measured by a Scanning Electron Microscopy/Energy Dispersive X-Ray Spectroscopy (SEM/EDS) and/or Scanning Electron Microscopy/Wavelength Dispersive Spectroscopy (SEM/WDS).

The method, apparatus, and system disclosed herein may be implemented in any means for achieving various aspects, and may be executed in a form of a non-transitory machine-readable medium embodying a set of instructions that, when executed by a machine, cause the machine to perform any of the operations disclosed herein. Other features will be apparent from the accompanying drawings and from the detailed description that follows.

### BRIEF DESCRIPTION OF FIGURES

The embodiments of this invention are illustrated by way of example and not limitation in the Figures of the accompanying drawings, in which like references indicate similar elements and in which:

FIG. 1 shows acid wood paper impregnated by pH indicator and deacidified (P1) and after superposing the drop or volume of water prescribed by a standard or preliminary tested and used for the particular type of porous material during the surface pH measurement (P2). The P1 sample is acid wood paper surface pH is 4.5 was impregnated by pH indicator methyl red, than deacidified by the immersion in suspension of MgO in perfluor heptane (Bookkeeper), for 10 seconds. P2 shows the same during pH measurement, immediately after a pH drop of water need for the measurement has been applied to the measured surface, according to one embodiment.

FIG. 2 shows seven longitudinal microscopic cross sections of supra-molecular structure of cellulose and paper, from cellulose macromolecule (on the right) to the sheet of paper (on the left), according to one embodiment. The read color indicate acid zone and yellow alkali pH zone. The dimensions shown are indicative, according to one embodiment.

FIG. 3 shows acid pH in the paper sheet micro-structure as visualized by pH methyl red indicator, according to one embodiment.

FIG. 4 shows MgO distribution in the acid paper (with original surface pH 5.3) deacidified by the suspension of MgO particles in perfluorheptane, having the average surface pH=10.2, according to one embodiment. The paper sample image thickness is h=60 μm; The size of the elementary measured area of interest—EMA selected in this case of observation and morphology analysis in the porous material cross section, the most important and critical direction for the efficacy of the neutralization and stabilization of acid paper and historical books, is EMA=10⁻¹-10⁻² μm². Impregnation: 10 minutes in the suspension of 4.3 g·l⁻¹ MgO in perfluoro heptane at laboratory temperature, according to one embodiment.

EMA may be only one elementary area of interest used for the measurement, scanning, and evaluation (similarly like pixel, px) in the whole image; Examples: EMA is shown in the FIG. 16C 6.

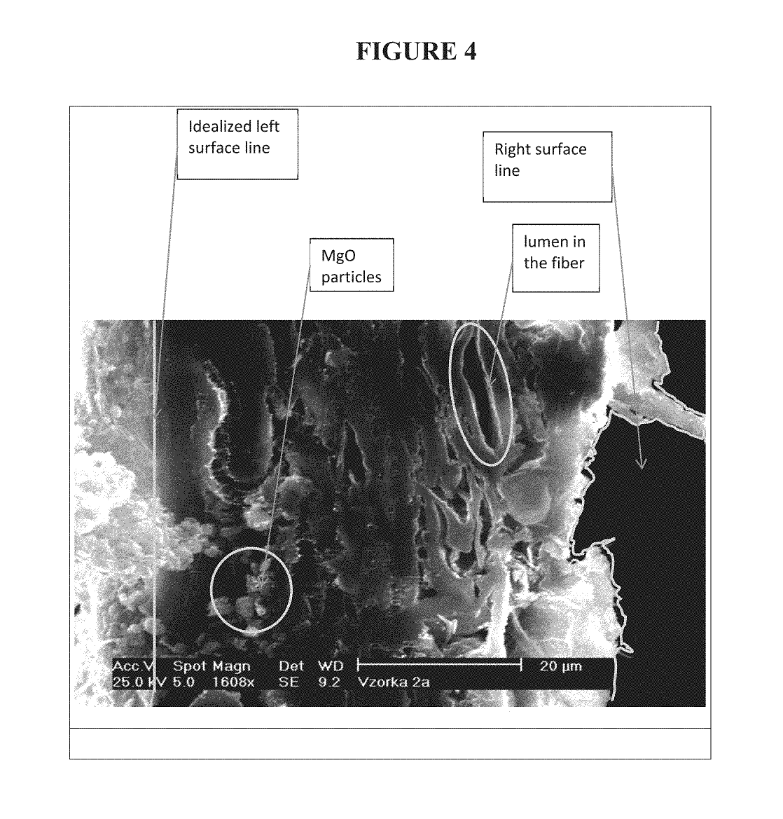

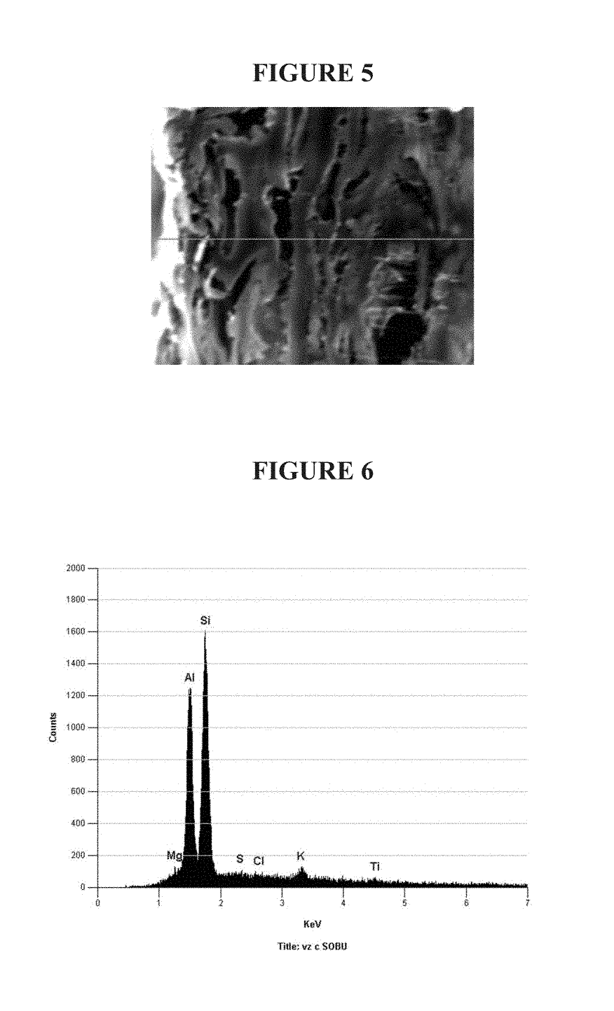

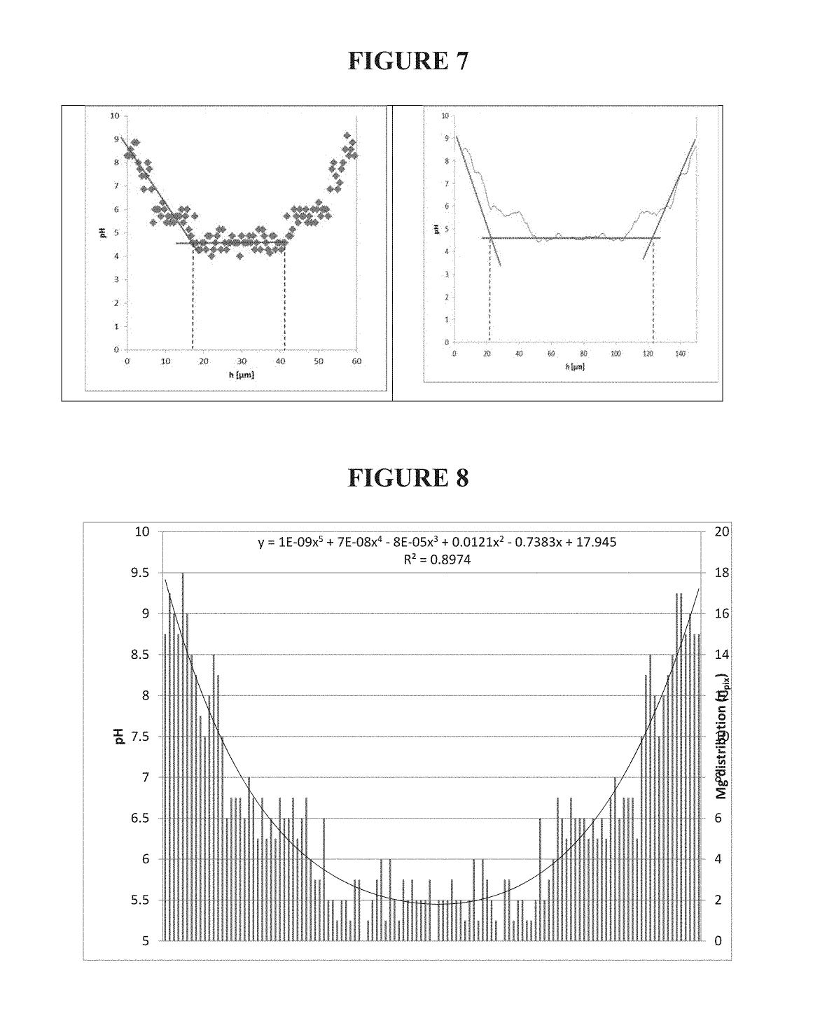

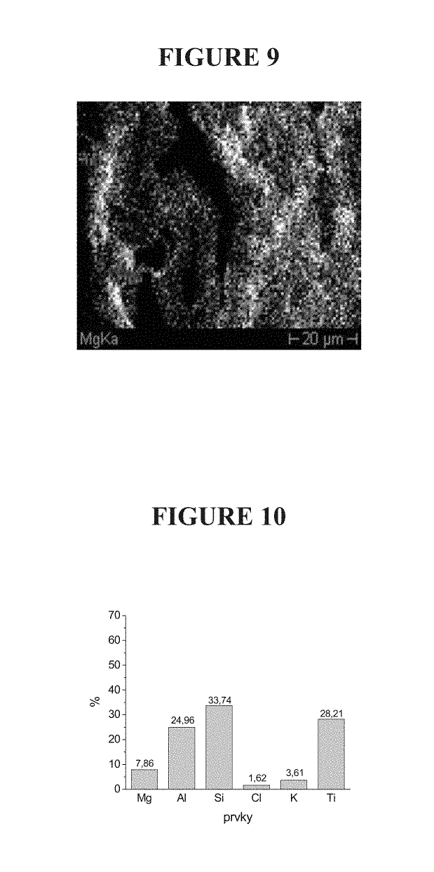

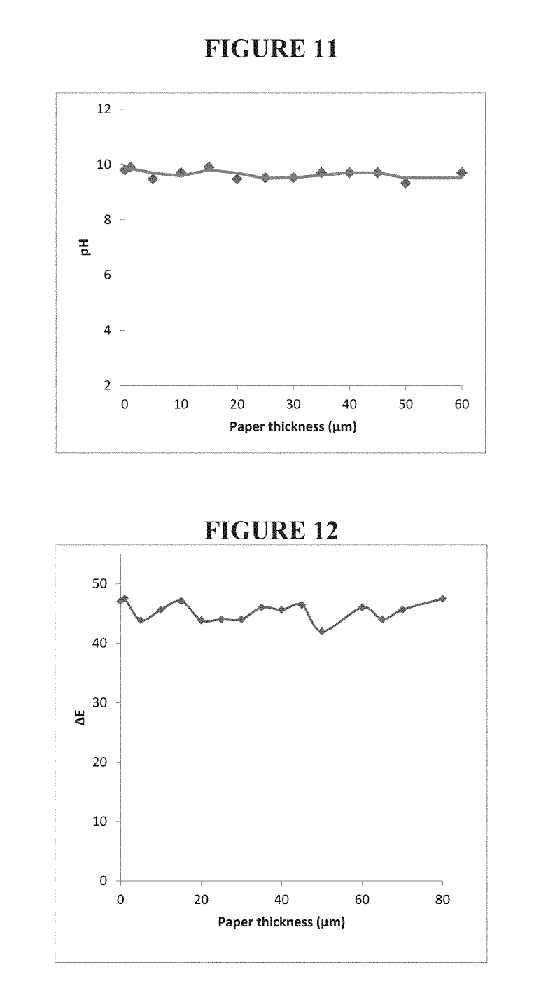

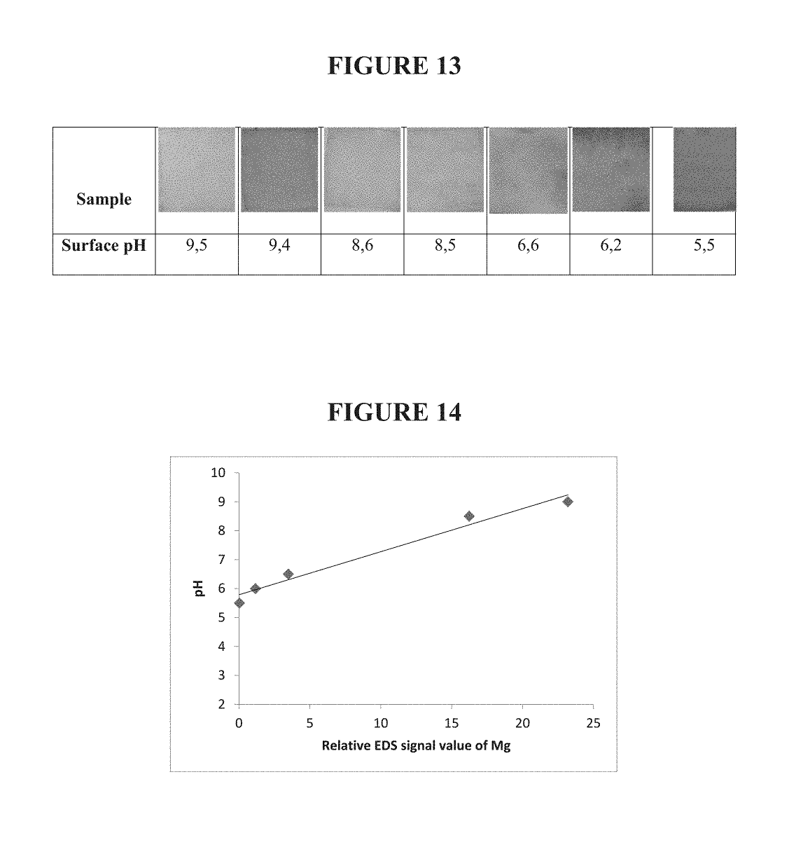

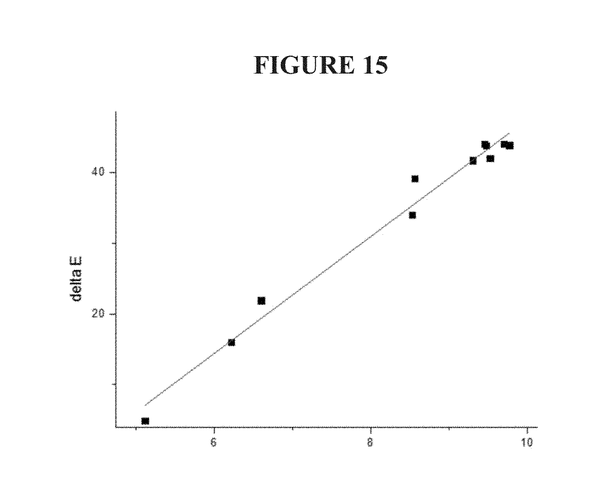

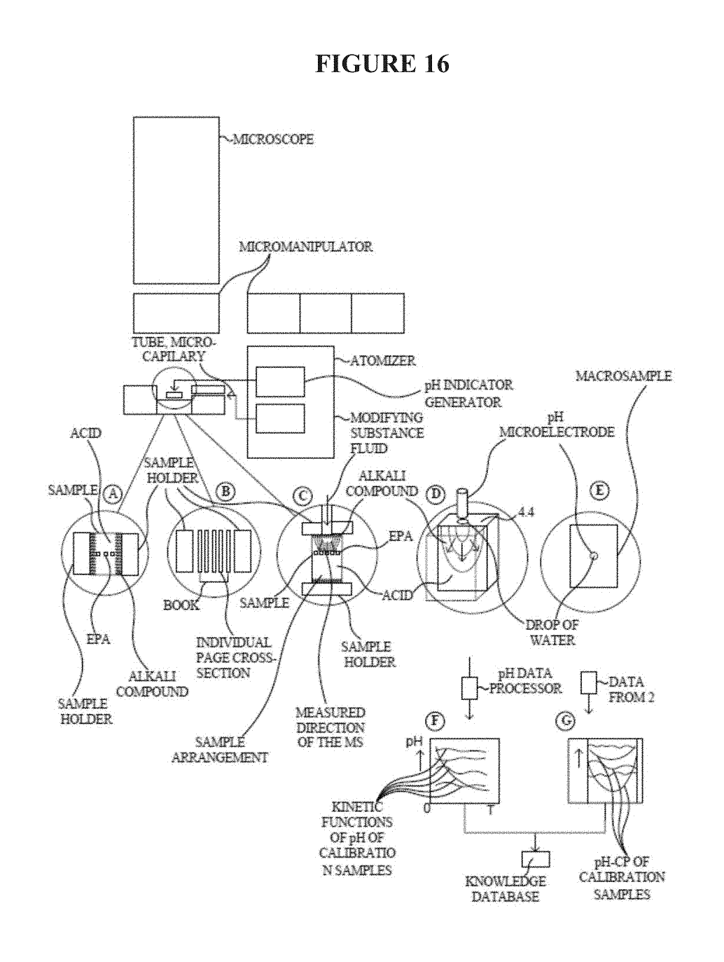

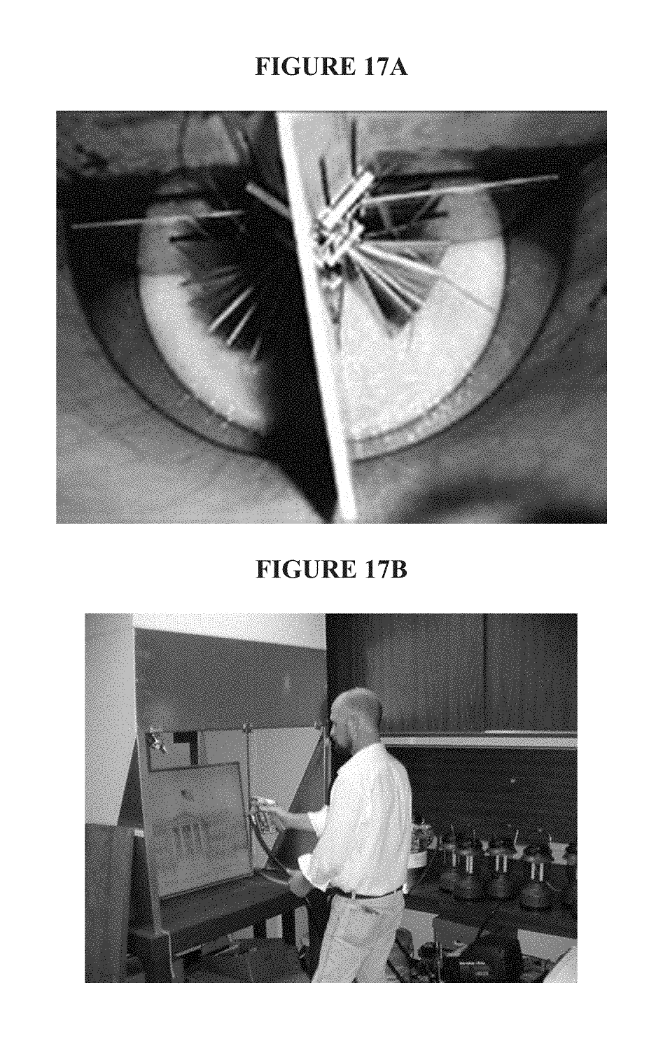

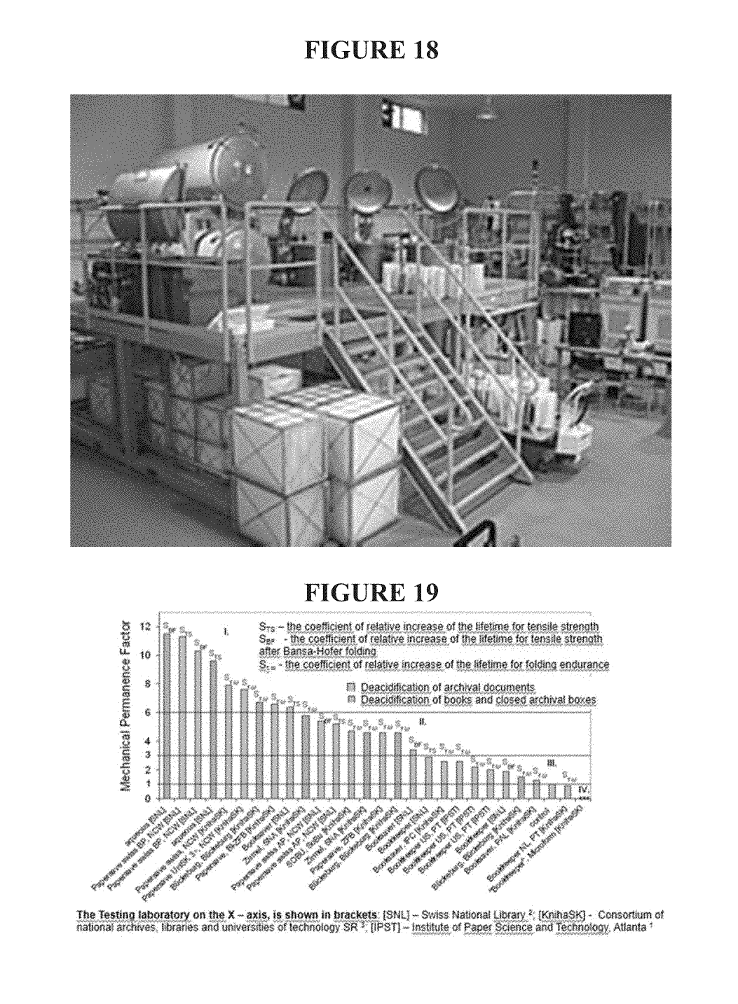

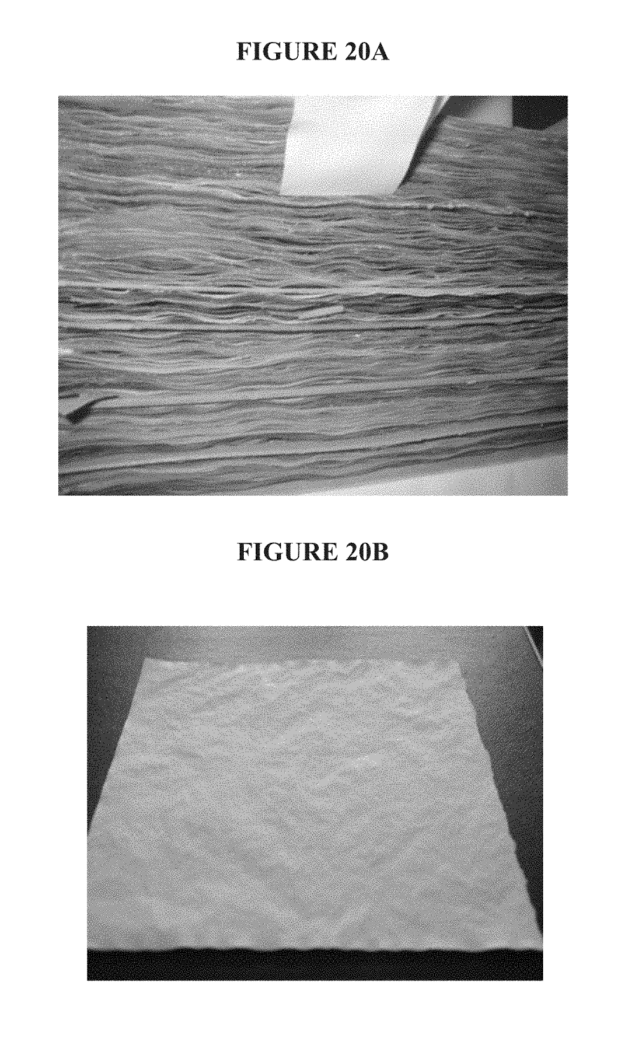

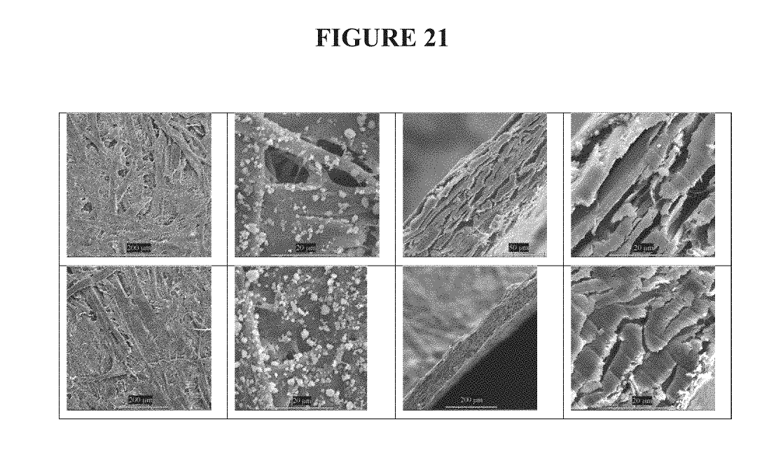

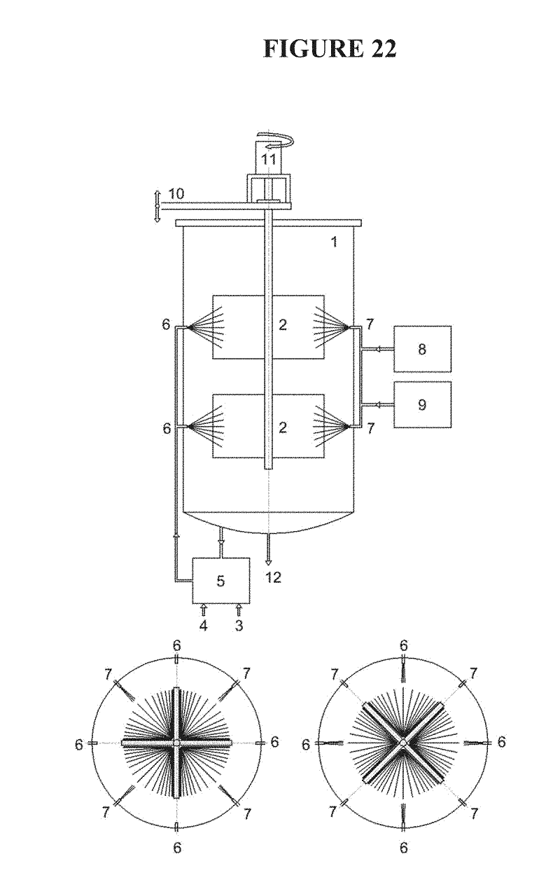

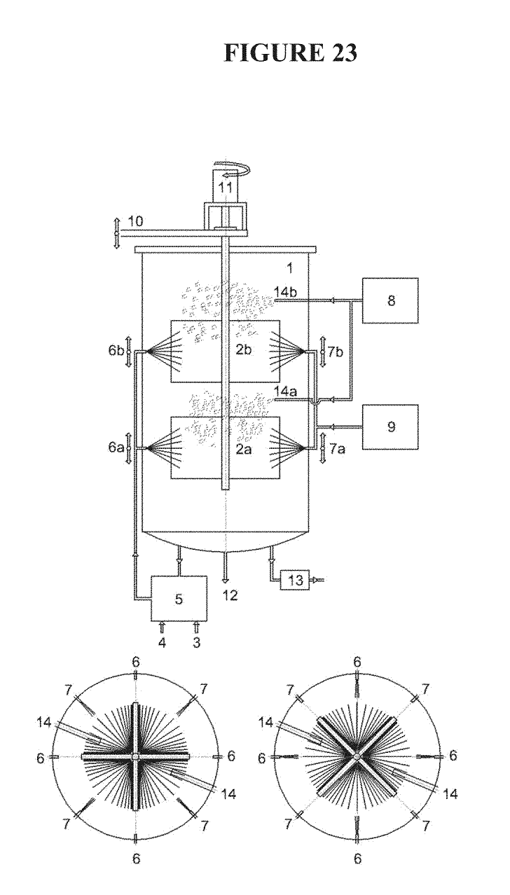

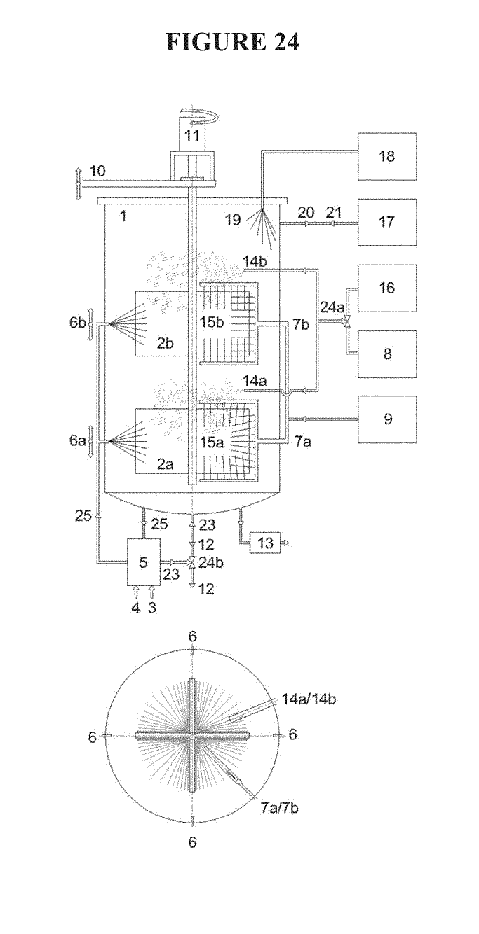

EMA description on the FIG. 4, on the page 6-7: “The size of the elementary measured area of interest—EMA selected in this case of observation and morphology analysis in the porous material cross section, the most important and critical direction for the efficacy of the neutralization and stabilization of acid paper and historical books, may be EMA=10-1-10-2 μm2 (while the whole microimage is 60×60 μm=3, 600 μm2 and EMA used for measurement distribution in FIGS. 7 and 8 was 60 μm2. The micro imaging of the sample containing the elementary measured areas may be performed.

FIG. 5 shows a SEM EDS of the paper sample modified by MgO particles in air (SoBu technology). Air conditioning in the air RH 50±1% at the temperature: 23.0±1° C., 24 hours, according to one embodiment.

FIG. 6 shows an EDS spectrum of Mg and other elements in the MgO particles deacidified paper (SoBu), according to one embodiment.

FIG. 7 shows two graphs of μpH distribution in the paper micro-structure cross-section expressed by linear model, according to one embodiment.

FIG. 8 shows a μpH polynomic distribution in the paper micro-structure using the sample shown in FIG. 5, according to one embodiment.

FIG. 9 shows the distribution of Mg in the cross section of a newsprint paper modified by the deacidification process used by Papersave Swiss NitroChem Wimmis Wimmis AG, according to one embodiment.

FIG. 10 shows a percentage of individual elements in a newsprint paper sample modified by the process Papersave Swiss NitroChem Wimmis Wimmis AG, according to one embodiment.

FIG. 11 shows distribution of the pH across the paper thickness (h) cross-section pH=f(h) using a sample of porous material, paper, deacidified with Mg(HCO₃)₂ aqueous solution 0.12 mol/l.

FIG. 12 shows calibration function between pH and total color difference ΔE, according to one embodiment.

FIG. 13 shows the relationship between color and pH achieved by impregnating acid paper with various aqueous Mg(HCO₃)₂ solution, according to one embodiment.

FIG. 14 shows the calibration curve pH=f(k_{Mg}×c_{Mg, EDS}), according to one embodiment.

FIG. 15 shows the calibration curve pH=f (ΔE), according to one embodiment.

FIG. 16 shows the apparatus that performs the method, according to one embodiment.

FIG. 17A shows a photo of an apparatus for deacidification books by alkaline particles in perfluorinated solvent.

FIG. 17B shows a photo of spraying technology using alkaline particles. (Photo by SK).

FIG. 18 shows the 8th mass deacidification plant in the world in Saitama City in March 2008. (Preservation Technologies Japan Office, 7-3-23 Ennami, Chuo-ku, Saitama City, Saitama 338-0007 Japan.)

FIG. 19 shows a comparative evaluation of mechanical permanence of deadified paper done with current methods and apparatuses known in the art.

FIGS. 20A and 20B shows the deformed paper after using a different method by deformed paper into a water solution using a Neschen apparatus and process.

FIG. 21 shows more samples deacidified by MgO particles (Bookkeeper); The 1st row of SEM images—deacidified acid paper from semipilot production (VUPC); The 2nd row deacidified industrial acid paper (SHP Slavosovce, a.s.). Airconditioning: humidity of air RH: 81%, temperature: 35, 0° C., time: 6 days.

FIG. 22 shows an apparatus for deacidifiying paper and porous materials.

FIG. 23 shows another apparatus for deacidifiying paper and porous materials.

FIG. 24 shows yet another apparatus for deacidifiying paper and porous materials.

Other features of the present embodiments will be apparent from the accompanying drawings and from the detailed description that follows.

### DETAILED DESCRIPTION

The following examples and description is provided to illustrate basic aspects of the methods of micro-pH measurement and of the estimating of the pH distribution in the micro-structure of porous or cell materials (pHd) described herein. Each example shows the most abundant cell and porous plant micro-structure, cellulose material. The cellulose material samples and micro-structures show wide range of both alkaline and acidic materials, a wide range of pH values, as well as, porous materials and their micro-structures with both homogeneous and heterogeneous pH distributions in submicroscopic views.

This disclosures develops new approaches that challenge the theory of neutralization and deacidification as commonly understood to one of skill in the art. Current implementations of neutralization and deacidification need improved methods, including pH measurement within the structure of porous materials. New, more sensitive methods are required to identify acid and alkaline parts in porous materials, and measure the differences between parts of the porous materials. Additionally, new methods are needed to measure, recognize and distinguish more precisely various types of acids, such as mineral and organic acids, present or arising in porous materials from processes used to produce the porous, use and future lifetime use, and accessory, idle, secondary, additional or side acids (AA) induced, activated or produced by any post production modification, stabilisation, conservation or protection such as deacidification in one of the most important porous and cell and cellulose materials.

The present disclosure relates to method and apparatus for measuring pH and micro-pH (μpH) distribution in porous materials, or cell or cellulose materials and objects such as books or paper documents intended to be saved for future generations, plant and food substance, biomaterials or biological materials or preparations. This invention is aimed also at identification of incomplete deacidification of books and paper.

One area of application of the methods described herein is plant or cellulose porous materials technology and stabilisation and conservation technology.

It may be that order to stop ongoing deacidification, users may need to neutralize acid at the micro-molecular level, in the micro-structure. Users, in many institutions, may use alkaline compounds that may not penetrate beneath the surface of a material.

Brittleness of many acid materials and objects may increase.

Disadvantages or problems of present neutralization and deacidification processes of acid porous materials are described. There are many problems and disadvantages of the storage of acid and deacidified books with irregular distribution of pH, alkaline and acid compounds in the material micro-structure.

There is reason to doubt that the widely used non-aqueous treatments, in which “alkaline reserve” particles deposited on the surface or in some void spaces of the paper, can achieve neutralization of acidity throughout the paper structure under the conditions most commonly used for treatment and storage. Alkaline particles such as CaCO₃, MgO, Mg(OH)₂, or ZnO can be present for long periods of time adjacent to acidic parts of cellulosic fibers without neutralization of the acidity, especially the acidity within the fibers.

Improper methods of measuring pH porous materials is the main reason why the incomplete neutralization methods still survive. They are used and spread worldwide.

Inadequate methods of measuring pH cannot recognize the acid places in the micro-structure of material with alkaline surface, nor discriminate between acidic and alkaline microscopic space. Thus, an even greater challenge exists because, at present, no prior method allows for a reliable quantification of pH, alkaline compounds deacidification reagents or alkaline reserve in the material micro-structure, especially over the material's micro-structure, which is the most critical dimension.

Some may not have an accurate method to test the true pH distribution in their materials, products or objects because they only test the pH at the macromolecular level. These users make decisions to stabilize acid materials, valuable cultural materials, documents, pictures, and other cultural objects based on incomplete, or incorrect information and methods “to increase the pH” of their acid product or object. This is because current methods of pH measurement known in the art provide data that is insufficient or false.

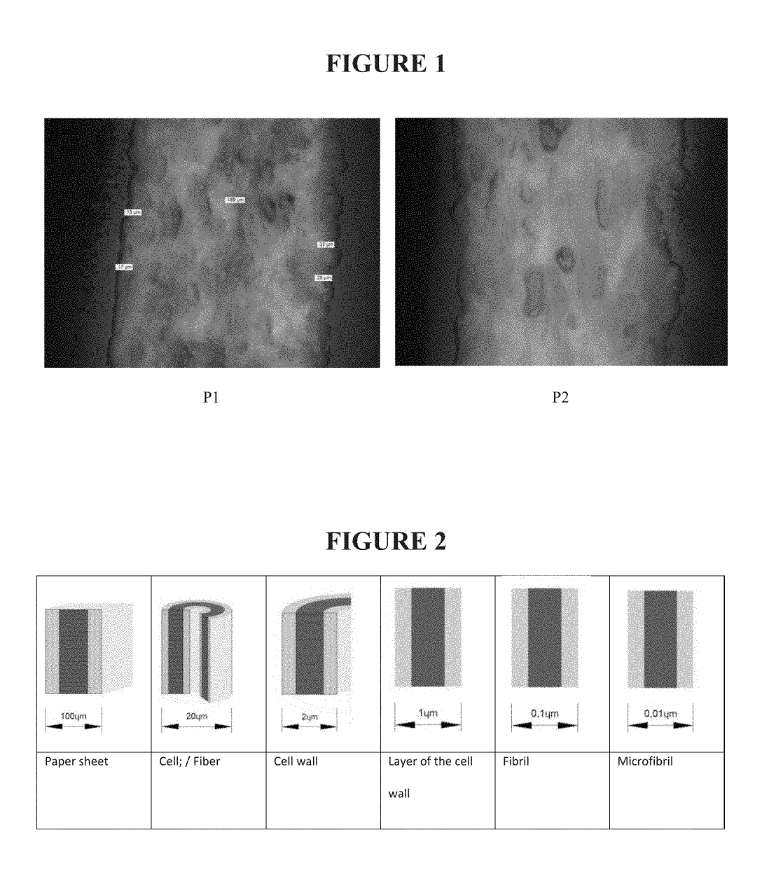

The measurement of pH in the traditional way uses necessary amount of water. The water cause unacceptable changes in pH redistribution of ions. Subsequently, giving inaccurate results. The main method of quality control processes neutralize the acid in the paper mass deacidification or alkaline paper production and is pH. Traditional methods of measuring the pH of the acid of interest cannot test the micro-structure of the fiber of the material. It can not identify acidic sites in the micro-structure of plant materials, paper and other cellulosic fibers. The main method for the measurement and quality control of the neutralization process pH measurement. As an example: The application of a droplet or larger amount of surface of the paper is necessary to measure the pH (FIG. 1, Example 1); If a drop of water applied to the surface of alkaline porous material, the material, the paper appears to be alkaline. This migration—secondary ion diffusion by the measuring drop of water causes the redistribution of alkali and acid ions inside the pore structure.

Traditional methods of pH measurement known in the art, use pH electrodes, microelectrodes, differential electrodes, pH indicators, extraction methods, which are unsuitable for micro-pH distribution measurements. These methods are often blind because they only take the surface pH. Data captured by surface pH methods give very limited information about the surface pH only. Surface pH measurements give very limited, if any information, about the pH, acidity, alkalinity of the sample measured, material or object, without knowing the pH distribution in the material, structure, micro-structure, or cross-sections.

Known methods of measuring pH of the material used for quality control and in quality management systems by memory institutions, can not distinguish between acid and alkaline macromolecules, neither between acidic or alkaline supramolecular structures in one seemingly alkaline material. The presently used methods of surface pH measurement or extraction methods, can not distinguish between hazardous acid and safer alkaline micro fibrils or fibrils. Used seen before and therefore can not distinguish or acidic and alkaline layers of cell wall, either acidic or alkaline lumens, neither even the whole acidic and alkaline cells. This means that the present methods do not distinguish acid fibres or paper fibers from alkaline. Or intra-fiber space in the paper. These methods actually do not distinguish large microscopic areas of the acidic paper in order 100×-1000× bigger places as the cellulose macromolecules.

The present methods are not able to distinguish between pH of completely acid-free paper and incompletely deacidified paper. In undiscovered acid fibers, cell walls, fibrils, microfibrils runs fast acid degradation. Hazardous statistical cleavage of the macromolecules of cellulose results in a rapid decrease of the polymerization degree, the reduction of fiber strength, increase the brittleness. Moreover the alkaline regions generate acids in both acidic and alkaline parts of micro-structure at higher rate than in acid paper.

Some improper pH measurement methods often produce false artifacts, and then measure the false artifacts generated by the measuring these false post-processing, post-production, post-conservation process, analytical method, and testing, evaluating, neither saying anything about the quality of the tested process, apparatuses, technology of production, stabilization, conservation, neutralization or deacidification of material.

Present methods and published, patented results have not quantified acids inside of so called “de-acidified”, or so called “alkaline” materials. In spite of that, they communicate on the “alkaline” materials, without knowing whether they are acid or alkaline. The methods known in the art have produced the results, discussions, communication and even conclusions on the “alkalinity”, “alkaline material”, on the “high pH of the measured sample” of materials, products and objects. In some cases, such results indicate a pH=8 to 11, despite the fact that 70-90% of the measured sample of material is acidic.

The pH data produced by unsuitable, non-relevant or false methods produce false results. This prevents users from seeking out adequate neutralization, deacidification, stabilization, conservation processes. It is a problem that needs to be solved.

The novel methods described herein allow for better pH, μpH and μpH distribution measurement in technology development, optimisation and Quality Management Control (QMS) of conservation processes of books, papers, plant materials, biomaterials, biologics, biological preparations, or other porous materials.

Each acidic, porous material shown in selected Examples described herein was prepared by pulping wood and making paper using acid alum process. The resulting alum-resin complex produces sulfuric acid, which creates an environment that creates oxidation and degradation reactions in the cell material of the paper, and a continuing oxidative environment producing organic carboxylic acids. The stability and quality of the materials and the properties of the products, or objects made from them, depend on the distribution of acid and alkaline compounds, alkaline reserve and pH in the supramolecular micro-structure. The explanation can be seen in Example 1, FIG. 1.

The following Examples using the method of the present invention is further described in detail.

### Example 1

Example 1 shows the problem and meaning of method and apparatus of micro-pH measurement in paper. These problems of acidity in micro-structure of paper is the cause of rapid degradation of cultural heritage of books. Example 1 further shows the disadvantages of the present “blind” methods of pH measurement. Existing methods may not detect the presence of acids inside the paper, and may not measure the pH distribution inside the paper (FIG. 1, P2). It also shows the evidence of uneven distribution of alkali and acids (FIG. 1).

As it can be seen from FIG. 1, sample P1 from the color change of the surface layers the penetration of alkali boundary to 7-17% from the paper thickness (FIG. 1, sample P1). P2 shown in FIG. 1 is the same sample during the pH measuring, immediately after the pH drop of water needed for the pH measurement has been applied to the measured surface.

Example 1 shows that in spite of the fact that about 80-90% of the paper thickness of the sample in FIG. 1 micro-structure is acid, the paper shown in P1, after the improper pH measurement looks neutralized, deacidified, alkaline, with pH value about 8-9. However, that is not the case. The cause for the erroneous pH indication is migration, migration is secondary diffusion, post-processing diffusion or any movement of a ion or a compound impregnated by primary intended penetration and diffusion into the final position in material or an object ready for use, or quality control. Improper analytical method is any method causing migration of the measured ions, either acid or alkali or other compounds, after the analysed and evaluated process.

As a result of the secondary diffusion by the drop of water used for analysis, the artifact (e.g., error which may be a misleading or confusing alteration in data or observation, for example in experimental science, which may result from flaws in technique or equipment) arises, and the whole paper appears alkali. This is the case even if the acids are present inside the cell structure. Such acid places are dangerous because they cause rapid statistical degradation of cellulose, increasing brittleness, rapidly decreasing longevity of the material. Without multiply splitting of paper layers used pH measuring methods fail to identify incomplete deacidification, acidic sites, cells, fibers, or the extent of acidic areas inside the paper or a book.

The smallest microprobe size available is larger than 100-150 micrometers when using the smallest pH micro-electrodes, and measurement spots exceed the whole thickness of a paper sheet. Moreover, the measuring spot is at least 300 micrometers large. The artifacts and false results caused by unsuitable pH measurement methods can arise at various sizes or levels of plant material micro-structure as shown in FIG. 2. Let us recognise here seven levels of structure shown in the descriptions of the cross section below.

The layers in FIG. 2 indicates possible pH distribution zones, with approximative order indicative dimensions; The read color indicate acid zone and yellow alkali pH zone. If the porous, cell, plant material, or cellulose materials are acid, they are stabilized by neutralization, alkalization or de-acidification processes. After these processes some part of the micro-structure can be alkaline, and the others still can stay acid. This situations are visualized in FIG. 2. For example in the micro-image signed as “Paper sheet”, we can see an sheet type of porous materials (such as paper) usually from 50 to 150 μm; in the first picture the average paper sheet 10² or 100 μm; The top layers is alkali and the inner is acid. The paper sheet consist of individual fibres, or cells, such as trachemys, or tracheids with the empty lumen inside; here again the outer and inner fibre surfaces can be alkaline (yellow), and the inner part of the substrate can still stay acid. Similarly visualized are smaller micro-structures with indicated potentially alkaline surface and acid inner parts.

### Example 2

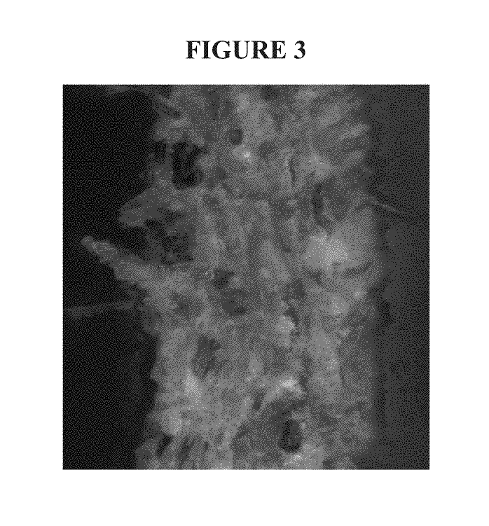

Example 2 shows the pH distribution measured by the methods described herein, for the samples of paper deacidified using MgO particles in perfluoroheptane. The sample of acid wood paper (NOVO, KLUG Conservation) surface pH 4.5 was impregnated by pH indicator methyl red, than deacidified by the immersion in suspension of MgO in perfluoroheptane. The aim of the measurement of micro-pH distribution in porous materials, or cell or cellulose materials and objects such as books or paper documents, and the problem formulation can be seen at FIG. 3. By various deacidification processes the alkali can diffuse into various parts of micro-structure of the material. Some parts of micro-structure are still acidic as indicated by the pink staining from the methyl red indicator (FIG. 3).

The Mg(c_{Mg,EDS}) was selected and used as pH characteristic property (CP_{M}) for the evaluation of pH distribution in this type of cellulose material, which were the samples of acid paper deacidified by MgO and the results are shown at FIG. 1-3.

The MgO distribution in the acid paper (with original surface pH 5.3) deacidified by the suspension of MgO particles in perfluoroheptane, having the average surface pH=10.2 can be seen at FIG. 3.

The result of the method, the pH distribution curves, can be seen at FIGS. 7, 8 and 9. The method is more clearly described as follows. Based on the series of preliminary measurements of potentially pH Characteristic Parameters (pH-CP) of cellulose materials the knowledge databases of potentially pH characteristic parameters have been found, and have been updated continually for new types of paper, other lignocellulosic materials, and various pH indicators. The pH of paper samples, color and other optical properties, SEM EDS (or WDS) signals corresponding to Mg, Al, Zn, Ca, K, Zr, R, G, B, CIE color parameters, and CIE Lab color parameters of paper macrosamples and their micro images were measured.

A Scanning Electron microscope (SEM) can reveal information about the paper sample external morphology (texture) and chemical composition, while the data are collected over a selected area of the surface of the sample, a 2-dimensional image is generated that displays spatial variations in these properties with magnification ranging from 20× to 30,000×, with resolution of 50 to 100 nm. The SEM using Energy Dispersive X-ray spectroscopy (EDS) or Wave Dispersive X-ray Spectroscopy (WDS) can analyse chemical compositions at selected points on the sample. As non-destructive method, it does not lead to volume loss of the sample, it can be used to analyze the same materials repeatedly. It gives information about the quantity and distribution of elemental composition within the micro-structure of the sample inside SEM with accuracy of 0.1-0.5%.

Based on this, the most Characteristic Parameters correlated well with the pH and alkaline compound concentration, were selected. The next requirement used for this evaluation is as follows: (1) the CP must also be invariant, or sensitive as low as possible, to factors of variability, influencing negatively accuracy and variability of results; (2) most important factors which should be eliminated using statistical methods were morphological structure factors as shown in the FIGS. 4 and 5, which show lumens, empty spaces or pores between cells, and fibres; (3) variable effects of microscopic sample surface structure (as seen in the right side surface of the sample in FIG. 4) negatively influencing color parameters variability, and other optical parameters; and (4) time, temperature, and more. We used standard statistical methods, namely sample selection, minimum number of samples of paper, and size of measured and evaluated image area required for the selected probability and reliability of measurement.

For the analysis, testing correlations between pH and pH-Characteristic Parameters (pH-CP), include, but are not limited to: (1) quantity and distribution of elemental composition, color and other spectral parameters of paper, or color of paper containing pH indicators; (2) selection of pH characteristic pH-CP, alkali elements—characteristic, or alkaline reserve characteristic parameters across the paper cross-sections—the EDS is suitable method; (3) if necessary to detect elements presented in very low concentrations, also WDS can be used as complementary technique with its increased sensitivity up to less than 0.02%; or (4) if necessary to distinguish between very close energies, to check for overlaps of the energy peaks, such as those shown in FIG. 7. FIG. 7 shows energy peaks for Magnesium (Mg), which is a characteristic paper deacidifying element, and Aluminum (Al), which is characteristic for the alum-rosin sizing, the most meaningful source of acidity in paper.

The results of testing and selecting pH-Characteristic Parameters (pH-CP), EDS concentration of Mg (c_{Mg,EDS}), Al (c_{Al,EDS}), the Mg (c_{Mg,EDS})/Al (c_{Al,EDS}) ratios, specific CIE partial color differences, the total color differences ΔE (CIE Lab), and others were found as suitable for alkali distribution measurement, alkaline reserve distribution measurement and pH distribution estimation in the micro-structure of this type of analysed cellulose material.

### Example 3

Example 3 shows the estimated pH distribution in a porous material, acid paper deacidified by MgO particles in nonpolar fluid. The linear model of the pH in the surface layer, combined with constant value line in the middle part of paper called paper core

As it can be seen the MgO particles are deposited mostly on the rough surface, and of course only in the pores connected with the surface larger than MgO particles.

In Example 3, the paper thickness of the paper sample is h=60 μm. The thickness of the broken line corresponding to the rugged rough, uneven paper surface is ca 10 μm, and the surface pores are larger than 1 micron from this broken surface line reach into more 10 μm; this corresponds to the total average thickness of the rugged and porous partially permeable surface layer containing some MgO particles is ca 20±5 μm. The size of the particles used for the deacidification was mostly smaller than 1 μm, and according to morphological analyses in the range 0.45 μm do 2.5 μm. The thickness of the paper on the image 3b is between 55 μm do 63 μm, and the pore size is from 0.2 μm to 2.5 μm.

The MgO particles can permeate into larger pores only if there is a route created to them from the surface. This transport goes approximately into the depth ca 20 μm of the paper cross-section. From the left edge of the paper, we can see the backward-facing surface of the paper. If a rough surface with a thickness of about 10 μm is not broken at some places, there is no transport of MgO particles at all to the interior of the pores, even if their size is larger than the particle size of MgO. In this case, the surface of deacidified paper is a filter layer through which only pure perfluoroheptane (solvent) penetrates. Therefore, MgO particles are deposited at the surface of such filtration layer.

FIG. 7, which shows the μpH distribution in the paper micro-structure cross section expressed by linear model: pH=k·h, where h is the paper most critical dimension—the paper cross section thickness, depicts two graphs. The graph on the left is μpH distribution in the partially deacidified and acid paper showin FIG. 5, with a thickness of the microsample h (ca 60 μm). The right graph presents the μpH distribution in the partially deacidified and acid paper with thickness h (ca 0.150 μm). The μpH distribution reflects also quantification of deacidification alkaline compounds (Aₗ) and partially alkaline reserve (Ar) distribution over the paper cross section; The similar SEM EDS or SEM WDS and calibration between the μpH, Aₗ and the Ar of calibration macro and microscopic samples enables simple quantifying also their distributions in the paper micro-structure.

The two linear distribution functions in FIG. 8 are connecting the both left and right alkaline surfaces (pH=10.2) with the acid paper core zone with pH=5.3.

When the paper thickness h=60 μm, the thickness of the broken line corresponding to the rugged rough, uneven paper surface is ca 10 μm, and the surface pores are larger than 1 micron from this broken surface line reach further 10 μm. The corresponding to the total average thickness of the rugged and porous partially permeable surface layer containing some MgO particles is ca 20±5 μm.

This paper contains both the left and right surfaces of the microscopic image similarly deacidified, with equal or similar surface values as measured by the surface pH electrode of pH˜10.

### Example 4

In this example it is shown that the polynomic function can be used as suitable model for the pH distribution in porous material—acid paper deacidified with MgO particles dispersion in perfluoroheptane.

The results are shown in FIG. 8, which shows a μpH polynomic distribution in the paper micro-structure using the sample from FIG. 4. The pH in various thickness (h) of the paper can be expressed by may be expressed or approximated by polynomic function pH=0.004h²−0.4931h+16,289, and R²=0.8835, connects both paper surfaces with pH=10.2 with the acid paper core zone with pH=5.3. The paper thickness h=60 μm.

The thickness of the broken line corresponding to the rugged rough, uneven paper surface is ca 5-10 μm, and the surface pores larger than 1 micron from this broken surface line reach further 10 μm, corresponding to the total average thickness of the rugged and porous partially permeable surface layer containing some MgO particles is ca 20±5 μm.

The pH of neutralized or deacidified paper depends mostly on distribution of alkaline and acidic elements and ions.

The acidic region with low pH is dangerous and critical in terms of the paper degradation, because of (A) extremely effective statistical degradation of cellulose macromolecules, (B) because of permanent diffusion of acid ions to the alkaline surface, and (C) the permanent diffusion resulting in accelerating of new additional acids generation inside the dangerous very rapid statistical degradation region initiated by the surface deacidification itself.

### Example 5

Example 5 shows the homogeneous distribution of pH samples impregnated by homogeneous solution of combination of magnesium alkoxide and titanium alkoxide in hexamethyldisiloxane, as well as other samples impregnated by water solutions of aqueous Mg(HCO₃)₂ solution.

In comparison with the non-even deacidification above (FIGS. 1 to 8) a more homogeneous distribution of Mg in the cross section of the newsprint paper was achieved when modified by the deacidification process (Papersave Swiss Nitrochemie Wimmis AG). The distribution of Mg in this paper is shown in FIG. 9, while the magnesium relative concentration is about 8% of what has been seen from the relevant percentage of individual elements shown in FIG. 10. FIGS. 9 and 10 show that the samples have more homogeneous pH distribution.

The example of homogeneous pH distribution in alkaline calibration sample prepared by impregnation of acid paper by aqueous Mg(HCO₃)₂ solution be seen in FIG. 11. Distribution of the pH across the paper thickness (h) cross-section pH=f(h); Example of porous material, paper, deacidified with deacidified with Mg(HCO₃)₂ aqueous solution. Relatively homogeneous pH distribution reflected by lower variability (higher egality) of color. The color variability expressed by the ΔE can be seen in the FIG. 13, where the ΔE is the total color difference between the color of an individual place on the surface, where the relevant surface pH value measured, and the average color of the original non-deacidified acid paper sample paper cross-section of deacidified acid paper.

FIG. 12 shows Calibration function between pH and total color difference ΔE as defined below.

### Example 6

This example shows the color of acid macroscopic samples of paper impregnated by pH indicator methyl red (FIG. 7, pH=4.3). It also shows the changes of color and pH changes achieved by impregnating acid of the paper samples with various aqueous Mg(HCO₃)₂ solutions. FIG. 13, shows how color relates to the pH, and it shows that the more acidic the pH, the brighter pink the sample becomes. This can be used for calibration. Example of calibration methods used in this invention for measurement of the distribution curves seen in FIG. 7, and FIG. 8 are shown in FIG. 14. The calibration curve pH=f(k_{Mg}×c_{Mg,EDS}) of FIG. 14 may be derived using this relationship. This shows Mg concentration as measured by the EDS c(Mg, EDS/WDS), expressed in EDS intensity units, for the selected elementary measured area (EMA)=60 aqueous Mg(HCO₃)₂ solution.

Symbols: k_{Mg} is the calibrating constant from the relationship between the relative EDS signal value of Mg, and the Mg concentration (c_{Mg}) as measured by any traditional method of elemental analysis; here we need and use the relative Mg concentration only expressed as relative EDS value of Mg concentration in paper.

For Example 6, FIG. 15 shows the calibration curve pH=f (ΔE). The ΔE is the total color difference between the color of an individual place on the surface, where the relevant surface pH value measured, and the average color of the original non-deacidified acid paper sample.

### Example 7

This example shows the apparatus of FIG. 16, which is used to take the μpH distribution measurement in material micro-structure. The apparatus comprises (1), aerosol generator, atomiser or nebulizer; (2) microscope, mobile microscope, smartphone microscope, microscope-spectrophotometer, microscope-colorimeter; (3) micro-apparatus and/or tools for preparation of the microscopic preparation.

More specifically the Apparatus of FIG. 16 comprises standard or special sample holder (5) for taking micro samples of books, or bound multipage documents micro sample cross-section, cross-sections of individual pages, sample arrangement for kinetic measurement of redistribution, homogenization, or deacidification completion; it also comprises pH microelectrodes, pH data processor, programs and database for storage and processing the pH kinetic data from calibration samples with known pH and pH distribution, kinetic functions of pH of calibration samples prepared by SAT or by other method of controlled impregnation of acid or neutral material by alkali studied/measured, data from microscope (2) calibration Database (DB) or knowledge database (KDB).

The legend for FIG. 16 is below.
1. Atomizer, jet nebulizer, aerosol generator
   1.1. pH indicator generator
   1.2. modifying substance, fluid, or drops generator (MS), such as alkaline solution aerosol generator, micro drops or nano drops generator; SAT (SK Patent 287856) generator
   1.3. tube, micro-capillary
2. Microscope, microscope-colorimeter, -photometer, -spectrophotometer;
3. Micromanipulator, micro scraper; (e.g., a micromanipulator may be a device which is used to physically interact with a sample under a microscope. Level of precision of movement is necessary that may not be achieved by the unaided human hand. It may consist of an input joystick, a mechanism for reducing the range of movement and an output section with the means of holding a micro tool to hold, inject, cut or otherwise manipulate the object as required. The mechanism for reducing the movement may require the movement to be free of backlash. This may be achieved by the use of kinematic constraints to allow each part of the mechanism to move only in one or more chosen degrees of freedom, which may achieve a high precision and repeatability of movement, usually at the expense of some absolute accuracy.)
4. Sample
   4.1. Book-, or bound multipage document—microsample cross-section
   4.2. Individual page cross-section
   4.3. Sample arrangement in 5 for kinetic measurement of redistribution, homogenization, or completion of 7 by 1.2
   4.4. Macroscopic sample
   4.5. Macrosample surface top view, with the measuring point and pH electrode (10); in the FIG. 16 C is visualized a microscope preparation prepared from the macroscopic sample 4.4
   4.6. Surface of 4.5
5. Sample holder
6. EMA (Elementary Measured Picture Area of interest, see claim 1)
7. Alkali compound diffusion from alkaline surface; or alkali part of the sample cross section visualization; or in the FIG. 16 C alkaline part of the surface of microscope preparation of sample 4 in the holder 5
8. Acid, or acid part of sample 4
9. Measured direction of the MS applied by the 1.3 from 1.2 onto the one spot, or larger surface of sample 4
10. pH microelectrode with a drop of water on the surface of 4.5
11. drop of water applied for the surface pH measurement by
12. pH data processor, pH kinetic data from calibration samples with known pH and pH distribution
13. kinetic functions of pH of calibration samples prepared by SAT or by other method of controlled impregnation of acid or neutral material by alkali studied/measured
14. Data from 2
15. pH-CP of calibration samples and other measured samples
16. Calibration Database (DB) or knowledge database (KDB).

Elementary Picture Area generally means: the smallest area of interest to evaluate pH distribution, alkaline compounds or alkaline reserve distribution; Area of interest of the observer, such as in morphological visual or image analysis evaluations of pH and CP; For example—to measure 100 μm in about minimally 10 steps the EMA could be in 10 μm in diameter or about 10¹ μm².

Characteristic Parameter measurement (CPₘ) generally means a sample of material of which the microscope samples can be prepared, such as paper sample 2×2 cm, thickness 50-200 microns. The macrosample is also used for measurements of calibration CP and kinetic CP. The dynamic pH characteristic parameters (CP_{t,pH}), or kinetics are measured as function of CP with time (t); for example the surface pH (pHₛ)=f(t), CIE color parameter, and total color difference ΔE or partial color differences of CIE, Helmhotz or other color parameters, or egality ΔEₛ, their relative indexes, ratios, differences, and their statistical parameters as function of time (t).

Characteristic parameter (CP) generally means a parameter of sample correlating with the measured pH, pH distribution (pHd), micro-pH (μpH) or micro-pH distribution (μpHd) in the micro-structure of the sample measured. CP may also mean possibly invariant on anything else but pH, and where the maximum correlates with pH and pH distribution.

Validation of measurements is critical to verifying validity, correctness and other statistical characteristics of the results is recommended and performed; this is especially useful for new type of material, and until the well validated calibration database or knowledge database are developed.

Subcritical Aqueous Technology (SAT) generally includes critical parameters controlled properties that must not be changed more than allowed by QMS (Quality Management System), which is deformation, color changes, chemical and physical properties changes, migration of chemicals, visible changes, etc. Applied to embodiments herein with respect to pH measurement. An important critical parameter is the pH and pH distribution, secondary diffusion or migration of pH related ions, metals such as Mg.

Sub-migration Cyclic Impregnation (SMI) of a sample in small safe steps, using necessary but safe amount of water-based solution of modifying substance (MS) in each step. Such solutions can be pH-indicator solution, or conservation water-based solutions, or water aerosols for dynamic μpHd measurement, enabling studying kinetics of the pH-distribution, measurement of dynamic pH characteristic parameters (CP_{t,pH}), homogenization of heterogeneous samples, or redistribution of pH in incompletely deacidified and conserved materials. The dynamic pH distribution measurements also enable measuring, optimizing and control of the samples from processes of air-conditioning of conserved or deacidified materials, strengthening or conservation or porous materials. This is micro mode of safe aqueous conservation technology that can be used for acid paper conservation.

SMI may be performed by impregnating/superpositioning of atomized water aerosols/mist in small safe steps, followed by a time for migration of substances inside, and then followed by drying the water, leaving the solid modifying substance/pH-indicator stay in the material. This cycle can be repeated carefully without causing any migration of ions without any unwanted change of the pH distribution measured, until retention of necessary amount of pH indicator, modifying substance is achieved.

One embodiment may be a method of measurement of pH distribution in the micro-structure of porous material, such as cell, plant, fiber or cellulose material, paper, such as test paper, test books (having no historical value, used for purposes of evaluating a method) impregnated with pH indicator, or cultural object of porous materials, comprising: (1) preparing and measuring microscopic sample at selected magnification and choosing the Elementary Picture Area of microimage area size of the micro-structure to be measured (EMA), with advantage from 0.1 to 5 μm.; (2) measuring pH characteristic parameters and pH of macrosamples (CP_{M}) and correlations between them, and choosing at least one CP, whereas the CP is a parameter of sample correlating with the measured pH, pH distribution (pHd), micro-pH (μpH) or micro-pH distribution (μpHd) in the micro-structure of the sample measured, and invariant, or possibly minimally depending on the other factors, the factors of variability; such factors of variability can be morphological structure, types of material, and other chemical or physical properties not correlating with pH; correlating with pH and pH distribution, such as Mg, Al, Zn, Ca EDS or WDS signals, color parameters or reflectance, or combination thereof; (3) calibration between the CP and pH using macroscopic samples; (4) calibration and/or validation between macrosample CP_{M} and microsample CPₘ images CPs at the selected magnification of interest (M), whereas the macrosample is a sample visible and measurable without using microscope, and microsample and micro-image and its EMA are invisible by free eye, and therefore can be seed and measured using optical or SEM microscope; (5) optionally (only when necessary or imprudent) running controlled safe micro version of sub-deformation subcritical Aqueous Technology (SAT), or Sub-migration Cyclic Impregnation (SMI) of the material using aqueous modifying substance solution such as pH indicator solution or metal indicator, whereas the SMI cycle comprise depositing the pH indicator solution aerosol or other to the surface of the material sample, with advantage using the apparatus according to claims 5-9 during, during the subcritical time tₛ and using subcritical amount of deposited water or aqueous solution at one cycle mₛ, followed by drying the material, or preparation; and (6) optionally followed by colorimetric control of the SMI using the measuring CP of EMA position, boundaries, and differences between two points, or lines ΔCP of EMA representing the pH distribution change by the pH measurement method itself, so that the maximum allowed migration is ΔCP=0−ΔCP_{crit}, and the submigration process is controlled by tₛ and mₛ.

Any embodiment of method of measurement described herein, wherein CP are concentrations of Mg, Al, measured by EDS or WDS.

A method of measurement of pH distribution within the micro-structure of a porous material, such as cell, plant, fiber or cellulose material, paper or cultural object of porous materials (next material), comprising the steps of:
a. preparing a microscopic sample of the porous material at a selected magnification;
b. selecting an elementary measured area of the microscopic sample or microimage;
c. microimaging the elementary measured area;
d. measuring one or more pH characteristic parameters and pH from the elementary measured area, and measuring a correlation between the one or more characteristic optical parameters from the elementary measured area and the pH of from the elementary measured area to obtain a correlated microscopic pH value and a microscopic characteristic optical parameter value and their distribution within the porous material micro-structure; whereas the pH characteristic parameter (CP) is a parameter of porous material microsample or macrosample correlating with the measured pH, pH distribution (pHd), micro-pH (μpH) or micro-pH distribution (μpHd) in the micro-structure of the sample measured, and invariant, or possibly minimally depending on the other factors of variability of pH, and pHd measurement which are to be eliminated or minimize; such factors of variability are porous morphological structure, defects, inter fibre or intra fibre pores, presence of lumens, sort of fibres, tissues inside, sort of material or raw materials used, such as sort of wood used for pulping, conservation process, whether the material was deacidified or not, modified, or otherwise treated, whether its average pH is alkaline or acid, and other chemical or physical properties not correlating with pH;
e. preparing a macrosopic sample of the porous material;
f. measuring and correlating one or more pH-characteristic parameters and pH of a macroscopic sample of the porous material to select the best correlating CP, and to obtain a correlated macroscopic pH value from the macroscopic pH characteristic optical parameter value; and
g. measuring and correlating one or more pH characteristic parameters and pH of the porous material microscope sample at various magnifications of interest to select the best correlating CP, and to obtain a correlated macroscopic pH value from the macroscopic pH characteristic optical parameter value.

The an elementary measured area may be approximately 0.1 to 5 microns.

Characteristic optical parameters may be selected from a group Magnesium-, Aluminium-, Zinc-, Calcium EDS signals, color parameters, CIE total color difference, reflectance, or a combination thereof.

The method may apply a subcritical no migration or sub-migration cyclic impregnation of the porous material using the aqueous solution of pH indicator, wherein the subcritical no migration or sub-migration cyclic impregnation further comprises depositing the pH indicator solution aerosol to the surface of the elementary measured area and macroscopic sample; and
a. applying colorimetric control of the subcritical, no migration or sub-migration cyclic impregnation by measuring one or more pH characteristic optical parameters at two different positions of sample to measure, control and eliminate the migration of alkali, acids of pH distribution, with advantage using the apparatus according to claims 5-9 during, the subcritical time tₛ and using subcritical amount of deposited water or aqueous solution at one cycle mₛ, followed by drying the material.

One or more characteristic pH characteristic optical parameters may be characterized or measured by SEM EDS or SEM WDS. One or more pH characteristic parameter may be characterized in the CIE tristimulárne or spectral characteristics of the optical properties in the visible spectrum of 400-700 nm of paper and/or pH indicator, indicating substance. Characterized in that the sample impregnation by pH indicator color by SAT is performed after the neutralization, deacidification conservation or the cell material.

An apparatus for measuring pH in micro-structure of a material, according to description above, wherein said apparatus comprises (1) atomiser or nebulizer, or aerosol generators; (2) a microscope, mobile or smartphone microscope; and (3) a micromanipulator with tools for preparation and modification of the microscopic preparation from porous materials, such as cultural material or object. The apparatus above in which tools is a cylindrical or rectangular blade for non-destructive or quasi non-destructive sampling, micro abrasion tool, and sample modification such as splitting, scanning and automated image analysis apparatus, or sheet splitting with a heat seal lamination technique apparatus.

An apparatus for the pH measurement in material micro-structure according to claim 1 comprising parts according to the Example 7. An apparatus for the pH measurement in material micro-structure according to claim 7 comprising parts according to the Example 7.

Any embodiment of method of measurement described herein, wherein CP is characterized by CIE tristimulus or spectral characteristics of the optical properties in the visible spectrum in the range of 400-700 nm of paper sample impregnated with pH-indicator.

Any embodiment of method of measurement described herein, characterized in that the sample impregnation by pH indicator color is performed after the neutralization, deacidification, conservation of the porous cell material by SAT using the apparatus 16.

Any embodiment of method of measurement, described herein characterized in that selected characteristic parameters CP correlating with the pH distribution are kinetic parameters (CP_{t,pH}) of surface pH measurement by surface pH electrode, such as kinetic constant, the initial pH value extrapolated to the kinetic measurement time zero, the pH value after stabilizing the pH values, ratios of pH values with the pH of calibration samples with known pH and distribution, and the statistical parameters.

Any embodiment of method of measurement described herein, characterized in that the microscope sample to be measured is freezed sample and then treated by pH-indicator solution at temperatures 15-60° C. by SAT or SMI using apparatus 16.

Any embodiment of method of measurement described herein, characterized in that the sample to be measured is embedded in polymer such as poly methyl methacrylate, in order to minimize altering of pH distribution, then the microscope preparation is placed to microscope of the apparatus (16), and the top layers are gradually removed from the surface in micromanipulator (3) of the apparatus (16), and the sample is then is sprayed or superposed by pH-indicator solution (1.1) at temperatures 15-60° C. by SAT or SMI using apparatus (16), and the distribution of CP on the sample surface (16A) is measured and transferred to database (16).

Another embodiment is an Apparatus for pH measurement in any material micro-structure using any method known in the art or described herein, wherein the apparatus comprises (1) aerosol generator, nebulizer or atomiser; (2) microscope, microscope-spectrophotometer, or SEM coupled to EDS or WDS, or mobile or smartphone microscopes similarly equipped; (3) apparatus or micromanipulator for preparation and modification of the microscopic preparation, with specialized tools for various porous materials such as microscraper, microabraser, freezer, lyofilizator, splitting analytical and testing apparatus for micro-pH distribution validation using splitting tissue calibrating materials into layers, and more.

Another embodiment is an Apparatus for pH measurement in material micro-structure according to any method described herein.

Another embodiment is a method of measurement of pH or μpH distribution in the micro-structure of porous material, such as cell, plant, fiber or cellulose material, paper, book or other cultural object of porous materials (next material), comprising the steps of: (1) measuring pH characteristic parameters (CP) and pH of samples at selected magnification (M) of interest (so that the morphological element of interest, such as paper cross section, cells or fibres are well visible); and their correlations with the pH using calibration samples (in the range of interest such as 4 to 11), and choosing at least one CP correlating with pH, whereas the potentially pH characteristic parameters can include absolute and relative values, the differential measurements CP, kinetics and kinetic pH characteristic parameters (CP_{t,pH}), and ratios, variability/egality of optical parameters of microscopic images, distribution functions and kinetic PCP from differential measurements of heterogeneous-homogeneous calibrating samples; model calibration equations between pH of calibration samples (CS), homogeneous CS, or CS with various types and degree of heterogeneity, whereas the CS are prepared by impregnation, such as safe subcritical aqueous techniques (SAT) of impregnation of acid paper by alkalic solutions, whereas the distribution type and depth of controlled by SAT impregnation time, concentration of alkali in the water solution and pH paper such as Mg, Al, Zn, Ca EDS signals, color parameters, reflectance, or combination thereof, using suitable statistical methods and models, or neural nets, creating calibration and knowledge database, evaluation and selection of the most pH characteristic parameters, and minimizing negative effects of factors of variability such as type of measured material, effect of morphology, negative optical effects in microscopy, transparency near the surface boundaries, opacity or gloss, evaluation by correlation and regression analysis between pH and potentially pH characteristic properties (PCP); (2) preparing and measurement of microscopic sample image at a selected magnification of interest, choosing suitable elementary picture area (of interest) microimage (EMA) size of the material micro-structure to be measured, with advantage from 0.1 to 10 microns size; (3) calibration between the CP and pH using macroscopic samples; and/or possibly comparison of calibrations between macro and micro image optical properties CP_{M} and CPₘ at the selected magnification (M), if necessary for validation and optimizing of the linear or non-linear calibration functions; and optionally (or alternatively) if the sample contains no pH indicator neither any suitable pH characteristic parameter (CP) such as color parameter-performing subcritical no migration or sub-migration cyclic impregnation (SMI) of the material with pH indicator solution, whereas one SMI cycle comprises depositing the pH indicator solution aerosol onto the surface of the material sample, with advantage using the apparatus according to claims 5-9 during a subcritical time tₛ and using subcritical amount of deposited water or aqueous solution at one cycle mₛ, followed by drying the material; whereas the tₛ and the mₛ, are estimated by the quantitative measurement of color migration of the color substances, their EMA or lines, using the macrosamples and microsamples containing pH indicator; (5) whereas the SMI using the measuring CP of EMA position, or differences between two points, or lines ΔCP of EMA representing the pH distribution change by the pH measurement method itself, so that the maximum allowed migration is ΔCOP=0 or <ΔCP_{crit}, and the submigration process is controlled by tₛ and mₛ; (6) elimination of lumens and other pores and empty spaces in cells, and cell material micro-structure, such as lumens, and interfibrous spaces by integration.

Another embodiment is a method for pH measurement is any method described herein, wherein the CP are the cation concentration of Mg, Al, measured by EDS of the measured picture cells, or the cell material.

Another embodiment is a method for pH measurement is any method described herein, wherein CP is/are the CIE tristimulus or spectral characteristics of the optical properties in the visible spectrum of 400-700 nm of paper containing pH or alkali compounds indicator, or other color indicating substance.

Another embodiment is a method for pH measurement is any method described herein, wherein characterized in that the sample impregnation by pH indicator color by SMI is performed after the neutralization, deacidification conservation or the cell material.

Another embodiment is an apparatus for the μpH measurement in material micro-structure according to claims 1 and 4 comprising (1) atomiser (2) microscope, or mobile or smartphone microscope (3) apparatus or tool for preparation of the microscopic preparation

Another embodiment is a method of measurement of pH distribution in the micro-structure of porous material, such as cell, plant, fiber or cellulose material, paper or cultural object of porous materials (next material), comprising the steps of (1) preparing and measurement of microscopic sample at selected magnification and choosing the elementary measured area of microimage (EMA) size of the micro-structure to be measured, with advantage from 0.1 to 5 microns; (2) measuring characteristic parameter and pH of macrosamples (CP_{M}) and their correlations, and choosing at least one COP correlating with pH, such as Mg, Al, Zn, Ca EDS signals, color parameters or reflectance, or combination thereof; (3) calibration between the and pH using macroscopic samples; (4) calibration between macro and micro image CP_{M} and CPₘ at the selected magnification (M); (5) if the sample does not contain pH indicator, neither well correlating characteristic CP than the measured sample of step (1) is treated by subcritical no migration or sub-migration cyclic impregnation (SMI) using either an aqueous or a non-aqueous solution of pH indicator, whereas the SMI cycle comprises depositing the pH indicator solution aerosol to the surface of the material sample, with advantage using the apparatus according to claims 5-9 during the subcritical time tₛ and using subcritical amount of deposited water or aqueous solution at one cycle mₛ, followed by drying the material; and (6) colorimetric control of the SMI using the measuring CP of EMA position, boundaries, and differences between two points, or lines ΔCP of EMA representing the pH distribution change by the pH measurement method itself, so that the maximum allowed migration is ΔCP=0−ΔCP_{crit}, and the submigration process is controlled by tₛ and mₛ.

According to the various embodiments described herein and in the various Figures, in one embodiment, a method of measurement of pH distribution within the micro-structure of a porous material, such as cell, plant, fiber or cellulose material, paper or cultural object of porous materials (next material) is disclosed. The method comprises the steps of (in any order):
j. preparing a microscopic sample of the porous material at a selected magnification;
k. selecting an elementary measured area of the microscopic sample or microimage;
l. microimaging the elementary measured area;
m. measuring one or more pH characteristic parameters and pH from the elementary measured area, and measuring a correlation between the one or more characteristic optical parameters from the elementary measured area and the pH of from the elementary measured area to obtain a correlated microscopic pH value and a microscopic characteristic optical parameter value and their distribution within the porous material micro-structure; whereas the pH characteristic parameter (CP) is a parameter of porous material microsample or macrosample correlating with the measured pH, pH distribution (pHd), micro-pH (μpH) or micro-pH distribution (μpHd) in the micro-structure of the sample measured, and invariant, or possibly minimally depending on the other factors of variability of pH, and pHd measurement which are to be eliminated or minimize; such factors of variability are porous morphological structure, defects, inter fibre or intra fibre pores, presence of lumens, sort of fibres, tissues inside, sort of material or raw materials used, such as sort of wood used for pulping, conservation process, whether the material was deacidified or not, modified, or otherwise treated, whether its average pH is alkaline or acid, and other chemical or physical properties not correlating with pH;
n. preparing a macroscopic sample of the porous material;
o. measuring and correlating one or more pH-characteristic parameters and pH of a macroscopic sample of the porous material to select the best correlating CP, and to obtain a correlated macroscopic pH value from the macroscopic pH characteristic optical parameter value; and
p. measuring and correlating one or more pH characteristic parameters and pH of the porous material microscope sample at various magnifications of interest to select the best correlating CP, and to obtain a correlated macroscopic pH value from the macroscopic pH characteristic optical parameter value.

An elementary measured area may be approximately 0.1 to 5 microns. Characteristic optical parameters may be selected from a group Magnesium-, Aluminum-, Zinc-, Calcium EDS signals, color parameters, CIE total color difference, reflectance, and/or a combination thereof.

The method may also include additional steps including, but not limited to:
q. applying a subcritical no migration or sub-migration cyclic impregnation of the porous material using the aqueous solution of pH indicator, wherein the subcritical no migration or sub-migration cyclic impregnation further comprises depositing the pH indicator solution aerosol to the surface of the elementary measured area and macroscopic sample; and
r. applying colorimetric control of the subcritical, no migration or sub-migration cyclic impregnation by measuring one or more pH characteristic optical parameters at two different positions of sample to measure, control and eliminate the migration of alkali, acids of pH distribution, with advantage using the apparatus according to claims 5-9 during, the subcritical time is and using subcritical amount of deposited water or aqueous solution at one cycle ms, followed by drying the material.

One or more characteristic pH characteristic optical parameters may be characterized and/or measured by Scanning Electron Microscopy/Energy Dispersive X-Ray Spectroscopy (SEM/EDS) and/or Scanning Electron Microscopy/Wavelength Dispersive Spectroscopy (SEM/WDS). One or more pH characteristic parameter may be characterized in the CIE tristimulárne or spectral characteristics of the optical properties in the visible spectrum of 400-700 nm of paper and/or pH indicator, indicating substance. The method may be characterized in that the sample impregnation by pH indicator color by SAT performed after the neutralization, deacidification, and conservation or the cell material.

An apparatus for measuring pH in micro-structure of a material may also be contemplated according to this aspect. The apparatus may include (1) atomizer or nebulizer, or aerosol generators; (2) a microscope, mobile or smartphone microscope; and (3) a micromanipulator with tools for preparation and modification of the microscopic preparation from porous materials, such as cultural material or object. The tools can be a cylindrical or rectangular blade for non-destructive or quasi non-destructive sampling, micro abrasion tool, and sample modification such as splitting, scanning and automated image analyses apparatus, or sheet splitting with a heat seal lamination technique apparatus. The apparatus for the pH measurement in material micro-structure in this aspect and others may comprise parts according to the Example 7.

According to the various embodiments described herein and in the various Figures, in another embodiment, a method of measurement of pH distribution within a micro-structure of a porous material includes: preparing a microscopic sample of the porous material at a selected magnification; selecting an elementary measured area of the microscopic sample, microimaging the elementary measured area, measuring a pH characteristic parameters (CP) and pH from the elementary measured area, measuring a correlation between the pH characteristic parameters from the elementary measured area and a pH of from the elementary measured area to obtain a correlated microscopic pH value and a microscopic characteristic optical parameter value and a distribution within a micro-structure of the porous material, whereas each of the pH characteristic parameters is a parameter of at least one of a porous material microsample and a porous material macrosample correlating with at least one of a measured pH, a pH distribution (pHd), a micro-pH (μpH), a micro-pH distribution (μpHd) in the micro-structure of the sample measured, an invariant, and possibly minimally depending on other factors of variability of pH, and pHd measurement which are an attempt to eliminate such factors of variability are porous morphological structure, defects, at least one of inter fibre and intra fibre pores, presence of lumens, sort of fibres, tissues inside, sort of material used, such as sort of wood used for pulping, conservation process, whether material was deacidified, modified, and otherwise treated, whether its average pH is alkaline, whether its average pH is acid, and other chemical and physical properties not correlating with pH.

The method in this other aspect also includes preparing a macroscopic sample of the porous material, measuring and correlating pH-characteristic parameters and pH of a macroscopic sample of the porous material to select the best correlating CP, and to obtain a correlated macroscopic pH value from a macroscopic pH characteristic optical parameter value, and measuring and correlating pH characteristic parameters and pH of the porous material microscope sample at various magnifications of interest to select the best correlating CP, and to obtain a correlated macroscopic pH value from the macroscopic pH characteristic optical parameter value. An elementary measured area may be approximately 0.1 to 5 microns.

According to the various embodiments described herein and in the various Figures, yet another embodiment, an apparatus to measure pH distribution within a micro-structure of a porous material includes a nebulizer to producing a fine spray of liquid, a microscope to magnify the porous material at least several hundred times, and a micromanipulator with a set of tools to prepare and modify a microscopic preparation from the porous material. The set of tools perform a set of functions including preparing a microscopic sample of the porous material at a selected magnification, selecting an elementary measured area of the microscopic sample, microimaging the elementary measured area, measuring a pH characteristic parameters (CP) and pH from the elementary measured area, measuring a correlation between the pH characteristic parameters from the elementary measured area and a pH of from the elementary measured area to obtain a correlated microscopic pH value and a microscopic characteristic optical parameter value and a distribution within a micro-structure of the porous material.

Each of the pH characteristic parameters is a parameter of at least one of a porous material microsample and a porous material macrosample correlating with at least one of a measured pH, a pH distribution (pHd), a micro-pH (μpH), a micro-pH distribution (μpHd) in the micro-structure of the sample measured, an invariant, and possibly minimally depending on other factors of variability of pH, and pHd measurement which are attempted to be eliminated such factors of variability are porous morphological structure, defects, at least one of inter fibre and intra fibre pores, presence of lumens, sort of fibres, tissues inside, sort of material used, such as sort of wood used for pulping, conservation process, whether material was deacidified, modified, and otherwise treated, whether its average pH is alkaline, whether its average pH is acid, and other chemical and physical properties not correlating with pH,

The set of tools may perform additional functions including preparing a macroscopic sample of the porous material, measuring pH-characteristic parameters and pH of a macroscopic sample of the porous material to select the best correlating CP, and to obtain a correlated macroscopic pH value from a macroscopic pH characteristic optical parameter value, measuring pH characteristic parameters and pH of the porous material microscope sample at various magnifications of interest to select the best correlating CP, and to obtain a correlated macroscopic pH value from the macroscopic pH characteristic optical parameter value, correlating pH-characteristic parameters and pH of the macroscopic sample of the porous material to select the best correlating CP, and to obtain the correlated macroscopic pH value from the macroscopic pH characteristic optical parameter value, and correlating pH characteristic parameters and pH of the porous material microscope sample at various magnifications of interest to select the best correlating CP, and to obtain a correlated macroscopic pH value from the macroscopic pH characteristic optical parameter value.

The nebulizer may be an aerosol generator and an atomizer to reduce the liquid into the fine spray. The set of tools may include a cylindrical and/or a rectangle blade for at least one of a non-destructive and a quasi non-destructive sampling, micro abrasion tool, and sample modification such as a splitting, a scanning and a automated image analysis apparatus, and sheet splitting with a heat seal lamination technique apparatus. An elementary measured area may be approximately 0.1 to 5 microns. The characteristic optical parameters may be selected from a group comprising at least one of a Magnesium-, Aluminum-, Zinc-, Calcium EDS signals, color parameters, CIE total color difference, reflectance, and a combination thereof.

The set of tools may perform a set of functions including applying at least one of a subcritical no migration and a sub-migration cyclic impregnation of the porous material using the aqueous solution of pH indicator, wherein the at least one of the subcritical no migration and the sub-migration cyclic impregnation further comprises depositing the pH indicator solution aerosol to the surface of the elementary measured area and macroscopic sample, and applying a colorimetric control of the subcritical, at least one of the no migration and the sub-migration cyclic impregnation by measuring pH characteristic optical parameters at two different positions of sample to measure, control and eliminate the migration of alkali, acids of pH distribution, with advantage using the apparatus according to claims 5-9 during, the subcritical time is and using subcritical amount of deposited aqueous solution at one cycle ms, followed by drying the material.

The characteristic pH characteristic optical parameters may be characterized and/or measured by a Scanning Electron Microscopy/Energy Dispersive X-Ray Spectroscopy (SEM/EDS) and/or Scanning Electron Microscopy/Wavelength Dispersive Spectroscopy (SEM/WDS).

The method, apparatus, and system disclosed herein may be implemented in any means for achieving various aspects, and may be executed in a form of a non-transitory machine-readable medium embodying a set of instructions that, when executed by a machine, cause the machine to perform any of the operations disclosed herein. Other features will be apparent from the accompanying drawings and from the detailed description that follows.

A number of embodiments have been described. Nevertheless, it will be understood that various modifications may be made without departing from the spirit and scope of the claimed invention. In addition, the logic flows depicted in the Figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

It may be appreciated that the various systems, methods, and apparatus disclosed herein may be embodied in a machine-readable medium and/or a machine accessible medium compatible with a data processing system (e.g., a computer system), and/or may be performed in any order.

The structures and modules in the Figures may be shown as distinct and communicating with only a few specific structures and not others. The structures may be merged with each other, may perform overlapping functions, and may communicate with other structures not shown to be connected in the Figures. Accordingly, the specification and/or drawings may be regarded in an illustrative rather than a restrictive sense.

What is claimed is:

1. A method of measurement of pH distribution within the micro-structure of a porous material, such as cell, plant, fiber or cellulose material, paper or cultural object of porous materials (next material),
comprising the steps of:
a. preparing a microscopic sample of the porous material at a selected magnification;
b. selecting an elementary measured area of the microscopic sample or microimage;
c. microimaging the elementary measured area:
d. measuring one or more pH characteristic parameters and pH from the elementary measured area, and measuring a correlation between the one or more characteristic optical parameters from the elementary measured area and the pH of from the elementary measured area to obtain a correlated microscopic pH value and a microscopic characteristic optical parameter value and their distribution within the porous material micro-structure; whereas the pH characteristic parameter (CP) is a parameter of porous material microsample or macrosample correlating with the measured pH, pH distribution (pHd), micro-pH (μpH) or micro-pH distribution (μpHd) in the micro-structure of the sample measured, and invariant, or possibly minimally depending on the other factors of variability of pH, and pHd measurement which are to be eliminated or minimize; such factors of variability are porous morphological structure, defects, inter fibre or intra fibre pores, presence of lumens, sort of fibres, tissues inside, sort of material or raw materials used, such as sort of wood used for pulping, conservation process, whether the material was deacidified or not, modified, or otherwise treated, whether its average pH is alkaline or acid, and other chemical or physical properties not correlating with pH;
e. preparing a macrosopic sample of the porous material;
f. measuring and correlating one or more pH-characteristic parameters and pH of a macroscopic sample of the porous material to select the best correlating CP, and to obtain a correlated macroscopic pH value from the macroscopic pH characteristic optical parameter value; and
g. measuring and correlating one or more pH characteristic parameters and pH of the porous material microscope sample at various magnifications of interest to select the best correlating CP, and to obtain a correlated macroscopic pH value from the macroscopic pH characteristic optical parameter value.

2. The method of claim 1, wherein the an elementary measured area is approximately 0.1 to 5 microns.

3. The method of claim 1, wherein characteristic optical parameters is selected from a group Magnesium-, Aluminium-, Zinc-, Calcium EDS signals, color parameters, CIE total color difference, reflectance, or a combination thereof.

4. The method of claim 1, further comprising the steps of:
a. applying a subcritical no migration or sub-migration cyclic impregnation of the porous material using the aqueous solution of pH indicator, wherein the subcritical no migration or sub-migration cyclic impregnation further comprises depositing the pH indicator solution aerosol to the surface of the elementary measured area and macroscopic sample; and
b. applying colorimetric control of the subcritical, no migration or sub-migration cyclic impregnation by measuring one or more pH characteristic optical parameters at two different positions of sample to measure, control and eliminate the migration of alkali, acids of pH distribution, with advantage using the apparatus according to claims 5-9 during, the subcritical time tₛ and using subcritical amount of deposited water or aqueous solution at one cycle mₛ, followed by drying the material.

5. The method of claim 1, wherein said one or more characteristic pH characteristic optical parameters are characterized or measured by SEM EDS or SEM WDS.

6. A method of claim 1, wherein said one or more pH characteristic parameter are characterized in the CIE tristimulãrne or spectral characteristics of the optical properties in the visible spectrum of 400-700 nm of paper and/or pH indicator, indicating substance.

7. A method of claim 1, characterized in that the sample impregnation by pH indicator color by SAT is performed after the neutralization, deacidification conservation or the cell material.

8. An apparatus for measuring pH in micro-structure of a material, according to claim 1 or 7, wherein said apparatus comprises (1) atomiser or nebulizer, or aerosol generators; (2) a microscope, mobile or smartphone microscope; and (3) a micromanipulator with tools for preparation and modification of the microscopic preparation from porous materials, such as cultural material or object.

9. An apparatus of claim 8, wherein said tools is a cylindrical or rectangular blade for non-destructive or quasi non-destructive sampling, micro abrasion tool, and sample modification such as splitting, scanning and automated image analysis apparatus, or sheet splitting with a heat seal lamination technique apparatus.

10. An apparatus for the pH measurement in material micro-structure according to claim 1 comprising parts according to the Example 7.

11. An apparatus for the pH measurement in material micro-structure according to claim 7 comprising parts according to the Example 7.

12. A method of measurement of pH distribution within a micro-structure of a porous material, comprising:
preparing a microscopic sample of the porous material at a selected magnification;
selecting an elementary measured area of the microscopic sample;
microimaging the elementary measured area;
measuring a pH characteristic parameters (CP) and pH from the elementary measured area;
measuring a correlation between the pH characteristic parameters from the elementary measured area and a pH of from the elementary measured area to obtain a correlated microscopic pH value and a microscopic characteristic optical parameter value and a distribution within a micro-structure of the porous material,
whereas each of the pH characteristic parameters is a parameter of at least one of a porous material microsample and a porous material macrosample correlating with at least one of a measured pH, a pH distribution (pHd), a micro-pH (μpH), a micro-pH distribution (μpHd) in the micro-structure of the sample measured, an invariant, and possibly minimally depending on other factors of variability of pH, and pHd measurement which are an attempt to eliminate such factors of variability are porous morphological structure, defects, at least one of inter fibre and intra fibre pores, presence of lumens, sort of fibres, tissues inside, sort of material used, such as sort of wood used for pulping, conservation process, whether material was deacidified, modified, and otherwise treated, whether its average pH is alkaline, whether its average pH is acid, and other chemical and physical properties not correlating with pH;
preparing a macroscopic sample of the porous material;
measuring and correlating pH-characteristic parameters and pH of a macroscopic sample of the porous material to select the best correlating CP, and to obtain a correlated macroscopic pH value from a macroscopic pH characteristic optical parameter value; and
measuring and correlating pH characteristic parameters and pH of the porous material microscope sample at various magnifications of interest to select the best correlating CP, and to obtain a correlated macroscopic pH value from the macroscopic pH characteristic optical parameter value.

13. The method of claim 1, wherein the an elementary measured area is approximately 0.1 to 5 microns.

14. An apparatus to measure pH distribution within a micro-structure of a porous material, comprising:
a nebulizer to producing a fine spray of liquid;
a microscope to magnify the porous material at least several hundred times; and
a micromanipulator with a set of tools to prepare and modify a microscopic preparation from the porous material,
wherein the set of tools to perform a set of functions comprising:
to prepare a microscopic sample of the porous material at a selected magnification,
to select an elementary measured area of the microscopic sample,
to microimage the elementary measured area,
to measure a pH characteristic parameters (CP) and pH from the elementary measured area,
to measure a correlation between the pH characteristic parameters from the elementary measured area and a pH of from the elementary measured area to obtain a correlated microscopic pH value and a microscopic characteristic optical parameter value and a distribution within a micro-structure of the porous material,
whereas each of the pH characteristic parameters is a parameter of at least one of a porous material microsample and a porous material macrosample correlating with at least one of a measured pH, a pH distribution (pHd), a micro-pH (μpH), a micro-pH distribution (μpHd) in the micro-structure of the sample measured, an invariant, and possibly minimally depending on other factors of variability of pH, and pHd measurement which are attempted to be eliminated such factors of variability are porous morphological structure, defects, at least one of inter fibre and intra fibre pores, presence of lumens, sort of fibres, tissues inside, sort of material used, such as sort of wood used for pulping, conservation process, whether material was deacidified, modified, and otherwise treated, whether its average pH is alkaline, whether its average pH is acid, and other chemical and physical properties not correlating with pH,
to prepare a macroscopic sample of the porous material,
to measure pH-characteristic parameters and pH of a macroscopic sample of the porous material to select the best correlating CP, and to obtain a correlated macroscopic pH value from a macroscopic pH characteristic optical parameter value,
to measure pH characteristic parameters and pH of the porous material microscope sample at various magnifications of interest to select the best correlating CP, and to obtain a correlated macroscopic pH value from the macroscopic pH characteristic optical parameter value,
to correlate pH-characteristic parameters and pH of the macroscopic sample of the porous material to select the best correlating CP, and to obtain the correlated macroscopic pH value from the macroscopic pH characteristic optical parameter value, and
to correlate pH characteristic parameters and pH of the porous material microscope sample at various magnifications of interest to select the best correlating CP, and to obtain a correlated macroscopic pH value from the macroscopic pH characteristic optical parameter value.

15. The apparatus of claim 14 wherein the nebulizer is at least one of an aerosol generator and an atomiser to reduce the liquid into the fine spray.

16. An apparatus of claim 14, wherein the set of tools comprise of at least one of a cylindrical and a rectangle blade for at least one of a non-destructive and a quasi non-destructive sampling, micro abrasion tool, and sample modification such as a splitting, a scanning and a automated image analysis apparatus, and sheet splitting with a heat seal lamination technique apparatus.

17. The apparatus of claim 14, wherein the an elementary measured area is approximately 0.1 to 5 microns.

18. The apparatus of claim 14, wherein the characteristic optical parameters is selected from a group comprising at least one of a Magnesium-, Aluminium-, Zinc-, Calcium EDS signals, color parameters, CIE total color difference, reflectance, and a combination thereof.

19. The apparatus of claim 14, wherein the set of tools to perform a set of functions further comprising:
to apply at least one of a subcritical no migration and a sub-migration cyclic impregnation of the porous material using the aqueous solution of pH indicator, wherein the at least one of the subcritical no migration and the sub-migration cyclic impregnation further comprises depositing the pH indicator solution aerosol to the surface of the elementary measured area and macroscopic sample, and
to apply a colorimetric control of the subcritical, at least one of the no migration and the sub-migration cyclic impregnation by measuring pH characteristic optical parameters at two different positions of sample to measure, control and eliminate the migration of alkali, acids of pH distribution, with advantage using the apparatus according to claims 5-9 during, the subcritical time tₛ and using subcritical amount of deposited aqueous solution at one cycle mₛ, followed by drying the material.

20. The apparatus of claim 14, wherein the characteristic pH characteristic optical parameters are at least one of characterized and measured by at least one of Scanning Electron Microscopy/Energy Dispersive X-Ray Spectroscopy (SEM/EDS) and Scanning Electron Microscopy/Wavelength Dispersive Spectroscopy (SEM/WDS).
